(19) United States
(12) Patent Application Publication
(10) Pub. No.: US 2010/0222363 A1
(43) Pub. Date: Sep. 2, 2010

(54) Bicyclic Derivatives as P38 Inhibitors

(75) Inventors: Carmen Almansa Rosales, Barcelona (ES); Marina Virgili Bernadó, Barcelona (ES)

Correspondence Address: ROTHWELL, FIGG, ERNST & MANBECK, P.C., 1425 K STREET, N.W., SUITE 800, WASHINGTON, DC, 20005, US

(73) Assignee: PALAU PHARMA, S.A., Palau-solità i Plegamans (ES)

(21) Appl. No.: 11/993,258

(22) PCT Filed: Jun. 28, 2006
(86) PCT No.: PCT/EP2006/006256

§ 371 Date: Dec. 20, 2007

(30) Foreign Application Priority Data
Jun. 29, 2005 (EP) 05380141.1

(51) Int. Cl. A61K 31/496; C07D 209/44; A61K 31/4035; C07D 405/02; C07D 401/02; A61K 31/454; C07D 403/02; A61P 25/00; A61P 9/00; A61P 19/08; A61P 29/00; A61P 31/00; A61P 37/00

(52) U.S. Cl. 514/254.08; 548/482; 514/416; 548/467; 514/414; 546/201; 514/323; 544/373

(57) ABSTRACT

New bicyclic derivatives of formula (I); wherein the meanings for the various substituents are as disclosed in the description. These compounds are useful as p38 kinase inhibitors.

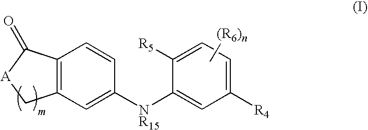

### FIELD OF THE INVENTION

The present invention relates to a new series of bicyclic derivatives, to processes to prepare them, to pharmaceutical compositions comprising these compounds as well as to their use in therapy.

### BACKGROUND OF THE INVENTION

Kinases are proteins involved in different cellular responses to external signals. In the Nineties, a new family of kinases called MAPK (mitogen-activated protein kinases) was discovered. MAPK activate their substrates by phosphorylation in serine and threonine residues.

MAPK are activated by other kinases in response to a wide range of signals including growth factors, pro-inflammatory cytokines, UV radiation, endotoxins and osmotic stress. Once they are activated, MAPK activate by phosphorylation other kinases or proteins, such as transcription factors, which, ultimately, induce an increase or a decrease in expression of a specific gene or group of genes.

The MAPK family includes kinases such as p38, ERK (extracellular-regulated protein kinase) and JNK (C-Jun N-terminal kinase).

p38 kinase plays a crucial role in cellular response to stress and in the activation pathway in the synthesis of numerous cytokines, especially tumor necrosis factor (TNF-α), interleukin-1 (IL-1), interleukin-6 (IL-6) and interleukin-8 (IL-8).

IL-1 and TNF-α are produced by macrophages and monocytes and are involved in the mediation of immunoregulation processes and other physiopathological conditions. For example, elevated levels of TNF-α are associated with inflammatory and autoimmune diseases and with processes that trigger the degradation of connective and bone tissue such as rheumatoid arthritis, osteoarthritis, diabetes, inflammatory bowel disease and sepsis.

Thus, it is believed that p38 kinase inhibitors can be useful to treat or prevent diseases mediated by cytokines such as IL-1 and TNF-α, such as the ones mentioned above.

On the other hand, it has also been found that p38 inhibitors inhibit other pro-inflammatory proteins such as IL-6, IL-8, interferon-γ and GM-CSF (granulocyte-macrophage colony-stimulating factor). Moreover, in recent studies it has been found that p38 inhibitors not only block cytokine synthesis but also the cascade of signals that these induce, such as induction of the cyclooxygenase-2 enzyme (COX-2).

Accordingly, it would be desirable to provide novel compounds which are capable of inhibiting the p38 kinase.

### DESCRIPTION OF THE INVENTION

One aspect of the present invention relates to the compounds of general formula I

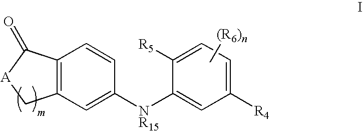

wherein:
A represents CR₁R₂ or NR₃;
R₁ and R₂ independently represent C₁₋₄ alkyl;
R₃ represents —(CH₂)ₚ—Cy¹, or C₁₋₆ alkyl optionally substituted with one or more R₇;
m represents 1 or 2;
R₄ represents —B—R₈;
R₅ represents hydrogen, C₁₋₄ alkyl, halogen or C₁₋₄ alkoxy;
R₆ can be attached to any available carbon atom of the phenyl ring and represents halogen or methyl;
n represents 0 or 1;
B represents —CONR₉—, —NR₉CO— or —NR₉CONR₉—;
R₇ represents hydroxy, C₁₋₄ alkoxy, halogen, —NR₁₀R₁₀ or phenyl optionally substituted with one or more groups selected from C₁₋₄ alkyl, halogen, C₁₋₄ alkoxy, C₁₋₄ haloalkyl and C₁₋₄ haloalkoxy, and additionally two R₇ groups on the same carbon atom can be bonded together to form a —(CH₂)_{q}— group;
R₈ represents C₁₋₆ alkyl or —(CH₂)ₚ—Cy²;
p represents 0, 1 or 2;
q represents 2, 3, 4, 5 or 6;
Cy¹ represents phenyl, heteroaryl, C₃₋₇ cycloalkyl or heterocyclyl, which can all be optionally substituted with one or more R₁₁;
Cy² represents phenyl, heteroaryl or C₃₋₇ cycloalkyl, which can all be optionally substituted with one or more R₁₂;
R₉ and R₁₀ independently represent hydrogen or C₁₋₄ alkyl;
R₁₁ represents halogen, R₁₃, —OR_{13′}, —NO₂, —CN, —COR_{13′}, —CO₂R_{13′}, —CONR_{14′}R_{14′}, —NR_{14′}R_{14′}, —NR_{14′}COR_{13′}, —NR_{14′}CONR_{14′}R_{14′}, —NR₁₄CO₂R₁₃, —NR_{14′}SO₂R₁₃, —SR_{13′}, —SOR₁₃, —SO₂R₁₃, —SO₂NR_{14′}R_{14′}, or Cy³;
R₁₂ represents C₁₋₄ alkyl, halogen, C₁₋₄ alkoxy, C₁₋₄ haloalkyl, C₁₋₄ haloalkoxy, or Cy³;
R₁₃ represents C₁₋₄ alkyl, C₁₋₄ haloalkyl or C₁₋₄ hydroxyalkyl;
R_{13′} represents hydrogen or R₁₃;
R₁₄ represents C₁₋₄ alkyl or C₁₋₄ hydroxyalkyl;
R_{14′} represents hydrogen or R₁₄;
Cy³ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl and C₁₋₄ haloalkoxy;
R₁₅ represents hydrogen, R₁₆, —COR₁₇, —CONHR₁₇, —SO₂R₁₇ or —COOR₁₇;
R₁₆ represents C₁₋₆ alkyl optionally substituted with one or more groups selected from halogen, —OR_{13′}, —NO₂, —CN, —COR_{13′}, —CO₂R_{13′}, —CONR_{14′}R_{14′}, —NR₁₈R₁₈, —NR_{14′}COR_{13′}, —NR_{14′}CONR_{14′}R_{14′}, —NR_{14′}CO₂R₁₃, —NR_{14′}SO₂R₁₃, —SR_{13′}, —SOR₁₃, —SO₂R₁₃, —SO₂NR_{14′}R_{14′} and Cy⁴;
R₁₇ represents R₁₆ or Cy⁴;
R₁₈ represents hydrogen, C₁₋₄ alkyl, C₁₋₄ hydroxyalkyl or C₁₋₄ alkoxyC₁₋₄alkyl;
Cy⁴ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl, C₁₋₄ haloalkoxy, hydroxy, C₁₋₄ hydroxyalkyl and —NR₁₉R₁₉; and
R₁₉ represents hydrogen or C₁₋₄ alkyl.

The present invention also relates to the salts and solvates of the compounds of formula I.

Some compounds of formula I can have chiral centres that can give rise to various stereoisomers. The present invention relates to each of these stereoisomers and also mixtures thereof.

The compounds of formula I are p38 kinase inhibitors and also inhibit the production of cytokines such as TNF-α.

Thus, another aspect of the invention relates to a compound of general formula I

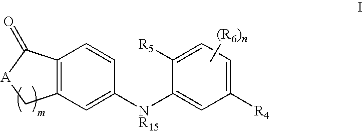

wherein:
A represents CR₁R₂ or NR₃;
R₁ and R₂ independently represent C₁₋₄ alkyl;
R₃ represents —(CH₂)ₚ—Cy¹, or C₁₋₆ alkyl optionally substituted with one or more R₇;
m represents 1 or 2;
R₄ represents —B—R₈,
R₅ represents hydrogen, C₁₋₄ alkyl, halogen or C₁₋₄ alkoxy;
R₆ can be attached to any available carbon atom of the phenyl ring and represents halogen or methyl;
n represents 0 or 1;
B represents —CONR₉—, —NR₉CO— or —NR₉CONR₉—;
R₇ represents hydroxy, C₁₋₄ alkoxy, halogen, —NR₁₀R₁₀ or phenyl optionally substituted with one or more groups selected from C₁₋₄ alkyl, halogen, C₁₋₄ alkoxy, C₁₋₄ haloalkyl and C₁₋₄ haloalkoxy, and additionally two R₇ groups on the same carbon atom can be bonded together to form a —(CH₂)_{q}— group;
Rₐ represents C₁₋₆ alkyl or —(CH₂)ₚ—Cy²;
p represents 0, 1 or 2;
q represents 2, 3, 4, 5 or 6;
Cy¹ represents phenyl, heteroaryl, C₃₋₇ cycloalkyl or heterocyclyl, which can all be optionally substituted with one or more R₁₁;
Cy² represents phenyl, heteroaryl or C₃₋₇ cycloalkyl, which can all be optionally substituted with one or more R₁₂;
R₉ and R₁₀ independently represent hydrogen or C₁₋₄ alkyl;
R₁₁ represents halogen, R₁₃, —OR_{13′}, —NO₂, —CN, —COR_{13′}, —CO₂R_{13′}, —CONR_{14′}R_{14′}, —NR_{14′}R_{14′}, —NR_{14′}COR_{13′}, —NR_{14′}CONR_{14′}R_{14′}, —NR_{14′}CO₂R₁₃, —NR_{14′}SO₂R₁₃, —SR_{13′}, —SOR₁₃, —SO₂R₁₃, —SO₂NR_{14′}R_{14′}, or Cy³;
R₁₂ represents C₁₋₄ alkyl, halogen, C₁₋₄ alkoxy, C₁₋₄ haloalkyl, C₁₋₄ haloalkoxy, or Cy³;
R₁₃ represents C₁₋₄ alkyl, C₁₋₄ haloalkyl or C₁₋₄ hydroxyalkyl;
R_{13′} represents hydrogen or R₁₃;
R₁₄ represents C₁₋₄ alkyl or C₁₋₄ hydroxyalkyl;
R_{14′} represents hydrogen or R₁₄;
Cy³ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl and C₁₋₄ haloalkoxy;
R₁₅ represents hydrogen, R₁₆, —COR₁₇, —CONHR₁₇, —SO₂R₁₇ or —COOR₁₇;
R₁₆ represents C₁₋₆ alkyl optionally substituted with one or more groups selected from halogen, —OR_{13′}, —NO₂, —CN, —COR_{13′}, —CO₂R_{13′}, —CONR_{14′}R_{14′}, —NR₁₈R₁₈, —NR_{14′}COR_{13′}, —NR_{14′}CONR_{14′}R_{14′}, —NR_{14′}CO₂R₁₃, —NR_{14′}SO₂R₁₃, —SR_{13′}, —SOR₁₃, —SO₂R₁₃, —SO₂NR_{14′}R_{14′} and Cy⁴;
R₁₇ represents R₁₆ or Cy⁴;
R₁₈ represents hydrogen, C₁₋₄ alkyl, C₁₋₄ hydroxyalkyl or C₁₋₄ alkoxyC₁₋₄alkyl;
Cy⁴ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl, C₁₋₄ haloalkoxy, hydroxy, C₁₋₄ hydroxyalkyl and —NR₁₉R₁₉; and
R₁₉ represents hydrogen or C₁₋₄ alkyl;
for use in therapy.

Another aspect of this invention relates to a pharmaceutical composition which comprises a compound of formula I or a pharmaceutically acceptable salt thereof and one or more pharmaceutically acceptable excipients.

Another aspect of the present invention relates to the use of a compound of formula I or a pharmaceutically acceptable salt thereof for the manufacture of a medicament for the treatment or prevention of diseases mediated by p38.

Another aspect of the present invention relates to the use of a compound of formula I or a pharmaceutically acceptable salt thereof for the manufacture of a medicament for the treatment or prevention of diseases mediated by cytokines.

Another aspect of the present invention relates to the use of a compound of formula I or a pharmaceutically acceptable salt thereof for the manufacture of a medicament for the treatment or prevention of diseases mediated by TNF-α, IL-1, IL-6 and/or IL-8.

Another aspect of the present invention relates to the use of a compound of formula I or a pharmaceutically acceptable salt thereof for the manufacture of a medicament for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to the use of a compound of formula I or a pharmaceutically acceptable salt thereof for the treatment or prevention of diseases mediated by p38.

Another aspect of the present invention relates to the use of a compound of formula I or a pharmaceutically acceptable salt thereof for the treatment or prevention of diseases mediated by cytokines.

Another aspect of the present invention relates to the use of a compound of formula I or a pharmaceutically acceptable salt thereof for the treatment or prevention of diseases mediated by TNF-α, IL-1, IL-6 and/or IL-8.

Another aspect of the present invention relates to the use of a compound of formula I or a pharmaceutically acceptable salt thereof for the treatment or prevention of a disease selected from immune, autoimmune and inflammatory diseases, cardiovascular diseases, infectious diseases, bone resorption disorders, neurodegenerative diseases, proliferative diseases and processes associated with the induction of cyclooxygenase-2.

Another aspect of the present invention relates to a method of treating or preventing a disease mediated by p38 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by cytokines in a subject in need thereof, especially a human being, which comprises administering to said subject a therapeutically effective amount of a compound of formula I or a pharmaceutically acceptable salt thereof.

Another aspect of the present invention relates to a method of treating or preventing a disease mediated by TNF-α, IL-1, IL-6 and/or IL-8 in a subject in need thereof, especially a human being, which comprises administering to said subject a therapeutically effective amount of a compound of formula I or a pharmaceutically acceptable salt thereof.

Another aspect of the present invention relates to a method of treating or preventing a disease selected from immune, autoimmune and inflammatory diseases, cardiovascular diseases, infectious diseases, bone resorption disorders, neurodegenerative diseases, proliferative diseases and processes associated with the induction of cyclooxygenase-2 in a subject in need thereof, especially a human being, which comprises administering to said subject a therapeutically effective amount of a compound of formula I or a pharmaceutically acceptable salt thereof.

Another aspect of the present invention relates to a process for the preparation of a compound of formula I as defined above, which comprises:

(a) when in a compound of formula I R₁₅ represents H, reacting a compound of formula IX with an amine of formula Xa

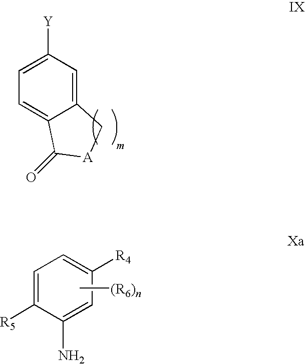

wherein A, R₄, R₅, R₆, m and n have the meaning described above and Y represents halogen or trifluoromethanesulfonate; or
(b) when in a compound of formula I R₄ represents —CONR₉R₈ and R₁₅ represents H, reacting a compound of formula II with an amine of formula HNR₈R₉ (III)

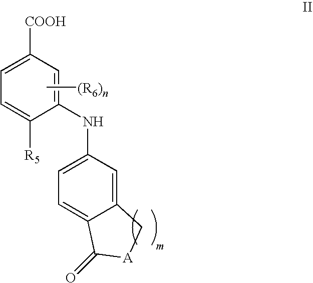

wherein A, R₅, R₆, R₈, R₉, m and n have the meaning described above; or
(c) when in a compound of formula I R₄ represents —NHCOR₈ and R₁₅ represents H, reacting a compound of formula IV with an acid of formula R₈COOH (V)

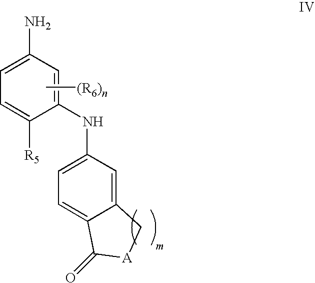

wherein A, R₅, R₆, R₈, m and n have the meaning described above; or
(d) when in a compound of formula I R₄ represents —NHCONHR₈, reacting a compound of formula IV with an isocyanate of formula R₈NCO (VI); or
(e) converting, in one or a plurality of steps, a compound of formula I into another compound of formula I.

In the above definitions, the term C₁₋ₙ alkyl, as a group or part of a group, means a straight or branched alkyl chain which contains from 1 to n carbon atoms. When n is 4, it includes the groups methyl, ethyl, propyl, isopropyl, butyl, isobutyl, sec-butyl and tent-butyl. When n is 6, examples include among others the groups methyl, ethyl, propyl, isopropyl, butyl, isobutyl, sec-butyl, tert-butyl, pentyl, isopentyl, neopentyl and hexyl.

A C₁₋₄ haloalkyl group means a group resulting from the replacement of one or more hydrogen atoms from a C₁₋₄alkyl group with one or more halogen atoms (i.e. fluoro, chloro, bromo or iodo), which can be the same or different. Examples include, among others, trifluoromethyl, fluoromethyl, 1-chloroethyl, 2-chloroethyl, 1-fluoroethyl, 2-fluoroethyl, 2-bromoethyl, 2-iodoethyl, 2,2,2-trifluoroethyl, pentafluoroethyl, 3-fluoropropyl, 3-chloropropyl, 2,2,3,3-tetrafluoropropyl, 2,2,3,3,3-pentafluoropropyl, heptafluoropropyl, 4-fluorobutyl and nonafluorobutyl.

A C₁₋₄ alkoxy group means an alkoxy group having from 1 to 4 carbon atoms, the alkyl moiety having the same meaning as previously defined. Examples include methoxy, ethoxy, propoxy, isopropoxy, butoxy, isobutoxy, sec-butoxy and tert-butoxy.

A C₁₋₄ haloalkoxy group means a group resulting from the replacement of one or more hydrogen atoms from a C₁₋₄ alkoxy group with one or more halogen atoms (i.e. fluoro, chloro, bromo or iodo), which can be the same or different. Examples include, among others, trifluoromethoxy, fluoromethoxy, 1-chloroethoxy, 2-chloroethoxy, 1-fluoroethoxy, 2-fluoroethoxy, 2-bromoethoxy, 2-iodoethoxy, 2,2,2-trifluoroethoxy, pentafluoroethoxy, 3-fluoropropoxy, 3-chloropropoxy, 2,2,3,3-tetrafluoropropoxy, 2,2,3,3,3-pentafluoropropoxy, heptafluoropropoxy, 4-fluorobutoxy and nonafluorobutoxy.

A C₁₋ₙ hydroxyalkyl group means a group resulting from the replacement of one or more hydrogen atoms from a C₁₋ₙ alkyl group with one or more hydroxy groups. Examples include, among others, hydroxymethyl, 1-hydroxyethyl, 2-hydroxyethyl, 1,2-dihydroxyethyl, 3-hydroxypropyl, 2-hydroxypropyl, 1-hydroxypropyl, 2,3-dihydroxypropyl, 4-hydroxybutyl, 3-hydroxybutyl, 2-hydroxybutyl and 1-hydroxybutyl.

A C₁₋₄ alkoxyC₁₋₄ alkyl group means a group resulting from the replacement of one hydrogen atom from a C₁₋₄ alkyl group with one C₁₋₄ alkoxy group such as those mentioned before. Examples include, among others, methoxymethyl, ethoxymethyl, propoxymethyl, isopropoxymethyl, butoxymethyl, isobutoxymethyl, sec-butoxymethyl, tert-butoxymethyl, 2-(methoxy)ethyl, 2-(ethoxy)ethyl, 2-(propoxy)ethyl, 2-(isopropoxy)ethyl, 2-(butoxy)ethyl, 3-(methoxy)propyl, 3-(ethoxy)propyl, and 4-(methoxy)butyl.

A halogen radical means fluoro, chloro, bromo or iodo.

A C₃₋₇ cycloalkyl group means a saturated monocyclic hydrocarbon ring having 3 to 7 carbon atoms, i.e. cyclopropyl, cyclobutyl, cyclopentyl, cyclohexyl and cycloheptyl.

The term heteroaryl means an aromatic 5- or 6-membered monocyclic or 8- to 12-membered bicyclic ring which contains from 1 to 4 heteroatoms selected from N, S and O. The heteroaryl group can be linked to the rest of the molecule through any available carbon or nitrogen atom. N atoms in the ring can be optionally oxidized forming N⁺O⁻. The heteroaryl group can be optionally substituted as disclosed above in the definitions of Cy¹, Cy², Cy³ and Cy⁴; if substituted, the substituents can be the same or different and can be placed on any available position in the ring. Examples of heteroaryl groups include among others 1,2,4-oxadiazolyl, 1,2,4-thiadiazolyl, 1,3,4-oxadiazolyl, 1,3,4-thiadiazolyl, furyl, imidazolyl, isoxazolyl, isothiazolyl, oxazolyl, pyrazolyl, pyrrolyl, thiazolyl, thienyl, 1,2,3-triazolyl, 1,2,4-triazolyl, pyrazinyl, pyridazinyl, pyridinyl, pyrimidinyl, benzimidazolyl, benzofuranyl, benzothiazolyl, benzothiophenyl, imidazopyrazinyl, imidazopyridazinyl, imidazopyridinyl, imidazopyrimidinyl, indazolyl, indolyl, isoindolyl, isoquinolinyl, naphthiridinyl, pyrazolopyrazinyl, pyrazolopyridinyl, pyrazolopyrimidinyl, purinyl, quinazolinyl, quinolinyl and quinoxalinyl.

A heterocyclyl group means a 3- to 7-membered monocyclic carbocyclic ring or an 8- to 12-membered bicyclic carbocyclic ring which can be saturated or partially unsaturated (i.e. non-aromatic) and which contains from 1 to 4 heteratoms selected from N, S and O, and wherein said ring can be linked to the rest of the molecule through any available carbon or nitrogen atom. Additionally, one or more C or S atoms in the ring can be optionally oxidized, forming CO, SO or SO₂ groups. The heterocyclyl group can be optionally substituted as disclosed above in the definitions of Cy¹, Cy³ and Cy⁴; if substituted, the substituents can be the same or different and can be placed on any available position in the ring. Preferably, the heterocyclyl is a 3- to 7-membered monocyclic ring. More preferably, the heterocyclyl ring has 5 or 6 ring atoms. Examples of heterocyclyl groups include, but are not limited to, aziridinyl, oxiranyl, oxetanyl, imidazolidinyl, isothiazolidinyl, isoxazolidinyl, oxazolidinyl, pyrazolidinyl, pyrrolidinyl, thiazolidinyl, dioxanyl, morpholinyl, piperazinyl, piperidinyl, pyranyl, tetrahydropyranyl, azepinyl, oxazinyl, oxazolinyl, pyrrolinyl, thiazolinyl, pyrazolinyl, imidazolinyl, isoxazolinyl, isothiazolinyl, tetrahydroisoquinolinyl, 2-oxo-pyrrolidinyl, 2-oxo-piperidinyl, 4-oxo-piperidinyl, 2-oxopiperazinyl, 2(1H)-pyridonyl, 2(1H)-pyrazinonyl, 2(1H)-pyrimidinonyl, 2(1H)-pyridazinonyl and phthalimidyl.

In the previous definition of heteroaryl, when the specified examples refer to a bicycle in general terms, all possible dispositions of the atoms are included. For example, the term pyrazolopyridinyl is to be understood as including groups such as 1H-pyrazolo[3,4-b]pyridinyl, pyrazolo[1,5-a]pyridinyl, 1H-pyrazolo[3,4-c]pyridinyl, 1H-pyrazolo[4,3-c]pyridinyl and 1H-pyrazolo[4,3-b]pyridinyl; the term imidazopyrazinyl is to be understood as including groups such as 1H-imidazo[4,5-b]pyrazinyl, imidazo[1,2-a]pyrazinyl and imidazo[1,5-a]pyrazinyl and the term pyrazolopyrimidinyl is to be understood as including groups such as 1H-pyrazolo[3,4-d]pyrimidinyl, 1H-pyrazolo[4,3-d]pyrimidinyl, pyrazolo[1,5-a]pyrimidinyl and pyrazolo[1,5-c]pyrimidinyl.

The expression “optionally substituted with one or more” means that a group can be substituted with one or more, preferably with 1, 2, 3 or 4 substituents, more preferably with 1 or 2 substituents, provided that said group has enough positions available susceptible of being substituted. When present, said substituents can be the same or different and can be placed on any available position.

In a compound of formula I, the group R₆ can be absent (n=0) or present (n=1). When R₆ is present, it can be placed on any available position on the phenyl ring.

When in a definition of a substituent two or more groups bearing the same numbering are shown (e.g. —NR₉CONR₉—, —NR₁₀R₁₀, —NR_{14′}CONR_{14′}R_{14′}, etc), this does not mean that they have to be identical. Each of them is independently selected from the list of possible meanings provided for that group, and therefore they can be the same or different.

The invention thus relates to the compounds of formula I as defined here above.

In another embodiment, the invention relates to compounds of formula I wherein A represents CR₁R₂.

In another embodiment, the invention relates to compounds of formula I wherein A represents NR₃.

In a further embodiment, the invention relates to compounds of formula I wherein m is 1.

In a further embodiment, the invention relates to compounds of formula I wherein m is 2.

In a further embodiment, the invention relates to compounds of formula I wherein A represents CR₁R₂ and m is 1.

In a further embodiment, the invention relates to compounds of formula I wherein A represents NR₃ and m is 1.

In a further embodiment, the invention relates to compounds of formula I wherein R₁ is identical to R₂.

In a further embodiment, the invention relates to compounds of formula I wherein R₁ is identical to R₂ and both represent methyl.

In a further embodiment, the invention relates to compounds of formula I wherein R₃ represents —(CH₂)ₚ—Cy¹, C₁₋₆ alkyl or C₁₋₆ hydroxyalkyl.

In a further embodiment, the invention relates to compounds of formula I wherein R₃ represents Cy¹, C₁₋₆ alkyl or C₁₋₆ hydroxyalkyl.

In a further embodiment, the invention provides compounds of formula I wherein R₃ represents Cy¹ or C₁₋₆ alkyl.

In a further embodiment, the invention provides compounds of formula I wherein Cy¹ represents C₃₋₇ cycloalkyl.

In a further embodiment, the invention relates to compounds of formula I wherein R₅ represents hydrogen, methyl, halogen or methoxy.

In a further embodiment, the invention relates to compounds of formula I wherein n is 0.

In a further embodiment, the invention relates to compounds of formula I wherein n is 0 and R₅ represents C₁₋₄ alkyl, halogen or C₁₋₄ alkoxy.

In a further embodiment, the invention relates to compounds of formula I wherein n is 0 and R₅ represents methyl, halogen or methoxy.

In a further embodiment, the invention relates to compounds of formula I wherein B represents —CONH—, —NHCO— or —NHCONH—.

In a further embodiment, the invention relates to compounds of formula I wherein B represents —CONR₉— or —NR₉CO—.

In a further embodiment, the invention relates to compounds of formula I wherein B represents —CONH— or —NHCO—.

In a further embodiment, the invention relates to compounds of formula I wherein B represents —CONR₉— and R₈ represents C₃₋₇ cycloalkyl.

In a further embodiment, the invention relates to compounds of formula I wherein B represents —CONH— and R₈ represents cyclopropyl.

In a further embodiment, the invention relates to compounds of formula I wherein R₁₅ represents hydrogen, R₁₆, —COR₁₇ or —SO₂R₁₇.

In a further embodiment, the invention relates to compounds of formula I wherein R₁₅ represents hydrogen, R₁₆ or —COR₁₇.

In a further embodiment, the invention relates to compounds of formula I wherein R₁₅ represents hydrogen or R₁₆.

In a further embodiment, the invention relates to compounds of formula I wherein R₁₅ represents hydrogen or C₁₋₆ alkyl optionally substituted with one or more groups selected from —OR_{13′}, —NR₁₈R₁₈ and Cy⁴.

In a further embodiment, the invention relates to compounds of formula I wherein R₁₅ represents hydrogen or C₁₋₆ alkyl optionally substituted with one group selected from —OR_{13′}, —NR₁₈R₁₈ and Cy⁴.

In a further embodiment, the invention relates to compounds of formula I wherein Cy⁴ represents Cy³ and —NR₁₈R₁₈ represents —NR_{14′}R_{14′}.

In a further embodiment, the invention relates to compounds of formula I wherein R₁₅ represents hydrogen or C₁₋₆ alkyl optionally substituted with one or more groups selected from −OR_{13′} and Cy³.

In a further embodiment, the invention relates to compounds of formula I wherein R₁₅ represents hydrogen.

Furthermore, the present invention covers all possible combinations of particular and preferred groups described hereinabove.

In a further embodiment, the invention relates to compounds according to formula I above which provide more than 50% inhibition of p38 activity at 10 μM, more preferably at 1 μM and still more preferably at 0.1 μM, in a p38 assay such as the one described in Example 22.

In a further embodiment, the invention relates to a compound according to formula I selected from:
N-Cyclopropyl-4-methyl-3-(1-oxo-2-phenyl-2,3-dihydroisoindol-5-ylamino)benzamide;
4,N-Dimethyl-3-(1-oxo-2-phenyl-2,3-dihydroisoindol-5-ylamino)benzamide;
N-Cyclopropyl-3-(2-ethyl-1-oxo-2,3-dihydroisoindol-5-ylamino)-4-methylbenzamide;
N-Cyclopropyl-3-[2-(3-hydroxypropyl)-1-oxo-2,3-dihydroisoindol-5-ylamino]-4-methylbenzamide;
N-Cyclopropyl-3-[2-(2-hydroxyethyl)-1-oxo-2,3-dihydroisoindol-5-ylamino]-4-methylbenzamide;
N-Cyclopropyl-3-(2,2-dimethyl-1-oxoindan-5-ylamino)-4-methylbenzamide;
N-Cyclopropylmethyl-3-(2,2-dimethyl-1-oxoindan-5-ylamino)-4-methylbenzamide;
4,N-Dimethyl-3-(2,2-dimethyl-1-oxoindan-5-ylamino)benzamide;
3-(2,2-Dimethyl-1-oxoindan-5-ylamino)-4-methyl-N-phenylbenzamide;
3-(2,2-Dimethyl-1-oxoindan-5-ylamino)-4-methyl-N-(3-pyridyl)benzamide;
N-Benzyl-3-(2,2-dimethyl-1-oxoindan-5-ylamino)-4-methylbenzamide;
3-(2,2-Dimethyl-1-oxoindan-5-ylamino)-4-methyl-N-(2-thiazolyl)benzamide;
3-(2,2-Dimethyl-1-oxoindan-5-ylamino)-4,N,N-trimethyl benzamide;
N-Cyclopropyl-3-(2,2-dimethyl-1-oxo-1,2,3,4-tetrahydronaphthalen-6-ylamino)-4-methylbenzamide;
N-[4-methyl-3-(1-oxo-2-phenyl-2,3-dihydroisoindol-5-ylamino)phenyl]furan-3-carboxamide;
2-Cyclopropyl-N-[4-methyl-3-(1-oxo-2-phenyl-2,3-dihydroisoindol-5-ylamino)phenyl]acetamide;
2-Cyclopropyl-N-[3-(2-(3-hydroxypropyl)-1-oxo-2,3-dihydroisoindol-5-ylamino)-4-methylphenyl]acetamide;
N-[3-(2,2-Dimethyl-1-oxo-1,2,3,4-tetrahydronaphthalen-6-ylamino)-4-methylphenyl]furan-3-carboxamide;
N-[3-(2,2-Dimethyl-1-oxoindan-5-ylamino)-4-methylphenyl]cyclopropylcarboxamide;
2-Cyclopropyl-N-[3-(2,2-dimethyl-1-oxoindan-5-ylamino)-4-methylphenyl]acetamide;
N-[3-(2,2-Dimethyl-1-oxoindan-5-ylamino)-4-methylphenyl]furan-3-carboxamide;
N-[3-(2,2-Dimethyl-1-oxoindan-5-ylamino)-4-methylphenyl]thiophene-2-carboxamide;
2-Chloro-N-[3-(2,2-dimethyl-1-oxoindan-5-ylamino)-4-methylphenyl]isonicotinamide;
N-[3-(2,2-Dimethyl-1-oxoindan-5-ylamino)-4-methylphenyl]-2-(pyrrolidin-1-yl)isonicotinamide;
2-Cyclopropyl-N-[3-(2,2-dimethyl-1-oxo-indan-5-ylamino)phenyl]acetamide;
N-[3-(2,2-Dimethyl-1-oxoindan-5-ylamino)-4-methylphenyl]acetamide;
N-[3-(2,2-Dimethyl-1-oxoindan-5-ylamino)-4-methylphenyl]-3-isopropylurea;
N-Cyclopropyl-3-[N-(2,2-dimethyl-1-oxoindan-5-yl)-N-methylamino]-4-methylbenzamide;
N-Cyclopropyl-3-[N-(2,2-dimethyl-1-oxoindan-5-yl)-N-(3-hydroxypropyl)amino]-4-methylbenzamide;
N-Cyclopropyl-3-[N-(2,2-dimethyl-1-oxoindan-5-yl)-N-(2-hydroxyethyl)amino]-4-methylbenzamide;
N-Cyclopropyl-3-[N-(2,2-dimethyl-1-oxoindan-5-yl)-N-(3-(morpholin-4-yl)propyl)amino]-4-methylbenzamide;
N-Cyclopropyl-3-[N-(2,2-dimethyl-1-oxoindan-5-yl)-N-(3-dimethylaminopropyl)amino]-4-methylbenzamide;
N-Cyclopropyl-3-[N-(2,2-dimethyl-1-oxoindan-5-yl)-N-(3-(4-(2-hydroxyethyl)-piperidin-1-yl)propyl)amino]-4-methylbenzamide;
3-[N-(3-(4-Aminopiperidin-1-yl)propyl)-N-(2,2-dimethyl-1-oxoindan-5-yl)amino]-N-cyclopropyl-4-methylbenzamide;
(R)—N-Cyclopropyl-3-[N-(2,2-dimethyl-1-oxoindan-5-yl)-N-(3-(3-hydroxypyrrolidin-1-yl)propyl)amino]-4-methylbenzamide;
N-Cyclopropyl-3-[N-(2,2-dimethyl-1-oxoindan-5-yl)-N-(3-(4-hydroxypiperidin-1-yl)propyl)amino]-4-methylbenzamide;
N-Cyclopropyl-3-[N-(2,2-dimethyl-1-oxoindan-5-yl)-N-(3-(2-methoxyethylamino) propypamino]-4-methylbenzamide;
N-Cyclopropyl-3-[N-(2,2-dimethyl-1-oxoindan-5-yl)-N-(3-(bis(2-hydroxyethyl)amino) propyl)amino]-4-methylbenzamide;
N-Cyclopropyl-3-[N-(2,2-dimethyl-1-oxoindan-5-yl)-N-[2-[(2-hydroxyethyl)methylamino]ethyl]amino]-4-methylbenzamide;
N-Cyclopropyl-3-[N-(2,2-dimethyl-1-oxoindan-5-yl)-N-(2-(piperazin-1-yl)ethyl)amino]-4-methylbenzamide;
N-Cyclopropyl-3-(2,2-dimethyl-1-oxoindan-5-ylamino)-4-fluorobenzamide;
N-Cyclopropyl-3-(2,2-dimethyl-1-oxoindan-5-ylamino)-4-methoxybenzamide;
4-Chloro-N-cyclopropyl-3-(2,2-dimethyl-1-oxoindan-5-ylamino)benzamide;
N-Cyclopropyl-3-(2,2-dimethyl-1-oxoindan-5-ylamino)benzamide;
2-Cyclopropyl-N-[5-(2,2-dimethyl-1-oxoindan-5-ylamino)-2-methylphenyl]acetamide;
N-Cyclopropyl-3-[N-(2,2-dimethyl-1-oxoindan-5-yl)-N-(2-methoxyacetyl)amino]-4-methylbenzamide;
3-[N-Cyclopropanecarbonyl-N-(2,2-dimethyl-1-oxoindan-5-yl)amino]-N-cyclopropyl-4-methylbenzamide;
3-(2-Cyclopentyl-1-oxo-2,3-dihydroisoindol-5-ylamino)-N-cyclopropyl-4-methylbenzamide; and
N-Cyclopropyl-3-[N-(2,2-dimethyl-1-oxoindan-5-yl)-N-(methanesulfonyl)amino]-4-methylbenzamide.

The compounds of the present invention may contain one or more basic nitrogens and may, therefore, form salts with organic or inorganic acids. Examples of these salts include: salts with inorganic acids such as hydrochloric acid, hydrobromic acid, hydroiodic acid, nitric acid, perchloric acid, sulfuric acid or phosphoric acid; and salts with organic acids such as methanesulfonic acid, trifluoromethanesulfonic acid, ethanesulfonic acid, benzenesulfonic acid, p-toluenesulfonic acid, fumaric acid, oxalic acid, acetic acid, maleic acid, ascorbic acid, citric acid, lactic acid, tartaric acid, malonic acid, glycolic acid, succinic acid and propionic acid, among others. Some of the compounds of the present invention may contain one or more acidic protons and, therefore, they may also form salts with bases. Examples of these salts include: salts with inorganic cations such as sodium, potassium, calcium, magnesium, lithium, aluminium, zinc, etc; and salts formed with pharmaceutically acceptable amines such as ammonia, alkylamines, hydroxylalkylamines, lysine, arginine, N-methylglucamine, procaine and the like.

There is no limitation on the type of salt that can be used, provided that these are pharmaceutically acceptable when they are used for therapeutic purposes. The term pharmaceutically acceptable salt represents those salts which are, according to medical judgement, suitable for use in contact with the tissues of humans and other mammals without undue toxicity, irritation, allergic response and the like. Pharmaceutically acceptable salts are well known in the art.

The salts of a compound of formula I can be obtained during the final isolation and purification of the compounds of the invention or can be prepared by treating a compound of formula I with a sufficient amount of the desired acid or base to give the salt in the conventional manner. The salts of the compounds of formula I can be converted into other salts of the compounds of formula I by ion exchange using ionic exchange resins.

The compounds of formula I and their salts may differ in some physical properties but they are equivalent for the purposes of the present invention. All salts of the compounds of formula I are included within the scope of the invention.

The compounds of the present invention may form complexes with solvents in which they are reacted or from which they are precipitated or crystallized. These complexes are known as solvates. As used herein, the term solvate refers to a complex of variable stoichiometry formed by a solute (a compound of formula I or a salt thereof) and a solvent. Examples of solvents include pharmaceutically acceptable solvents such as water, ethanol and the like. A complex with water is known as a hydrate. Solvates of compounds of the invention (or salts thereof), including hydrates, are included within the scope of the invention.

Some of the compounds of the present invention may exist as several diastereoisomers and/or several optical isomers. Diastereoisomers can be separated by conventional techniques such as chromatography or fractional crystallization. Optical isomers can be resolved by conventional techniques of optical resolution to give optically pure isomers. This resolution can be carried out on any chiral synthetic intermediate or on products of general formula I. Optically pure isomers can also be individually obtained using enantiospecific synthesis. The present invention covers all individual isomers as well as mixtures thereof (for example racemic mixtures or mixtures of diastereomers), whether obtained by synthesis or by physically mixing them.

The compounds of formula I can be obtained by following the processes described below. As it will be obvious to one skilled in the art, the exact method used to prepare a given compound may vary depending on its chemical structure. Moreover, in some of the processes described below it may be necessary or advisable to protect the reactive or labile groups by conventional protective groups. Both the nature of these protective groups and the procedures for their introduction or removal are well known in the art (see for example Greene T. W. and Wuts P. G. M, “Protective Groups in Organic Synthesis”, John Wiley & Sons, 3^{rd} edition, 1999). As an example, as protective groups of an amino function tert-butoxycarbonyl (Boc) or benzyl (Bn) groups can be used. The carboxyl groups can be protected for example in the form of C₁₋₄ alkyl esters or arylalkyl esters, such as benzyl, while the hydroxyl groups can be protected for example with tetrahydropyranyl (THP) or benzyl (Bn) groups. Whenever a protective group is present, a later deprotection step will be required, which can be performed under standard conditions in organic synthesis, such as those described in the above-mentioned reference.

Unless otherwise stated, in the methods described below the meanings of the differents substituents are the meanings described above with regard to a compound of general formula I.

Compounds of formula I wherein R₄═—CONR₉R₈ and R₁₅═H (Ia) can be obtained from a compound of formula II and an amine of formula III, as shown in the following scheme:

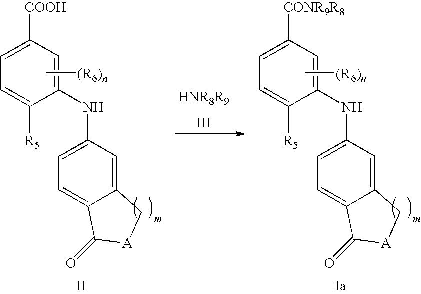

wherein A, R₅, R₆, R₈, R₉, m and n have the meaning described above. This reaction is carried out in the presence of an activating agent such as (benzotriazol-1-yloxy)tripyrrolidinophosphonium hexafluorophosphate, N-(3-dimethylaminopropyl)-N′-ethylcarbodiimide hydrochloride or N,N-dicyclohexylcarbodiimide and 1-hydroxybenzotriazol, and in the presence of a base such as N,N-diisopropylethylamine or N-methylmorpholine and in a suitable solvent such as dimethylformamide. Alternatively, the reaction can be carried out by conversion of the carboxylic acid of formula II into an acyl chloride, by using standard conditions in organic synthesis, followed by conversion of the latter into the amide of formula Ia by reaction with an amine of formula III in the presence of a base such as triethylamine, in a suitable solvent such as for example dichloromethane, and cooling, preferably at 0° C.

Compounds of formula I wherein R₄═—NHCOR₈ and R₁₅═H (Ib) can be obtained from a compound of formula IV and an acid of formula V, as shown in the following scheme:

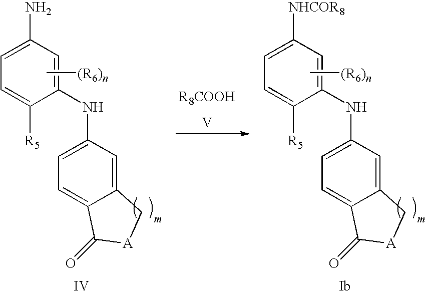

wherein A, R₅, R₆, R₈, m and n have the meaning described above. This reaction is carried out under the same conditions described above for the preparation of compounds Ia from compounds II and III.

The compounds of formula I wherein R₄═—NHCONR₉R₈ and R₁₅═H (Ic) can be obtained from a compound of formula IV, as shown in the following scheme:

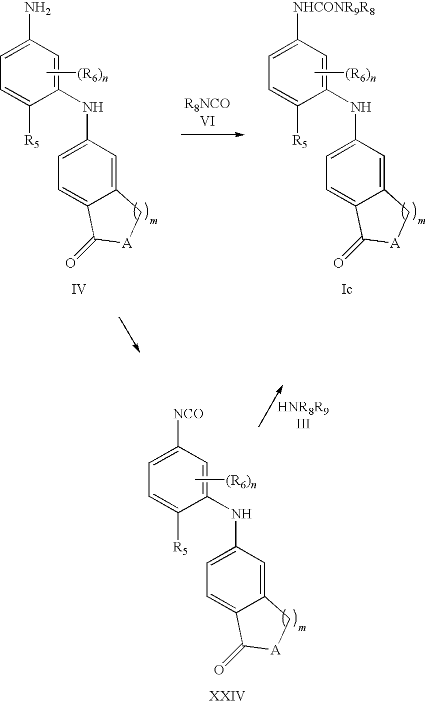

wherein A, R₅, R₆, R₈, R₉, m and n have the meaning described above. The compounds of formula Ic wherein R₉═H can be obtained by treatment of a compound IV with an isocyanate of formula VI. This reaction is carried out in a suitable solvent, such as dimethylformamide, and at a suitable temperature comprised between room temperature and the temperature of the boiling point of the solvent. Alternatively, a compound of formula Ic can be obtained from a compound of formula IV by a two step sequence which involves converting the amine into the corresponding isocyanate (XXIV) with triphosgene, in the presence of a base such as N,N-diisopropylethylamine, triethylamine or N-methylmorpholine, in a suitable solvent such as acetonitrile or a halogenated hydrocarbon such as chloroform or dichloromethane; and then reacting the resulting isocyanate XXIV with an amine of formula III in a suitable solvent, such as the solvent used in the first step.

Compounds of formula II can be obtained by hydrolysis of esters of formula VII; as shown in the following scheme:

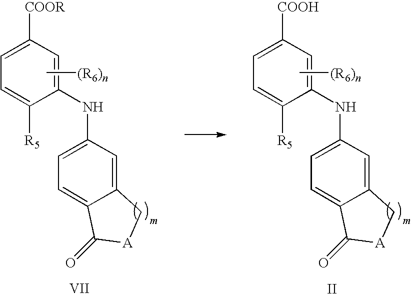

wherein R represents C₁ alkyl and A, R₅, R₆, m and n have the meaning described above. This reaction can be carried out in the presence of a base, such as KOH, in a suitable solvent such as ethanol, and preferably heating.

Compounds of formula IV can be obtained by reduction of nitro compounds of formula VIII, as shown in the following

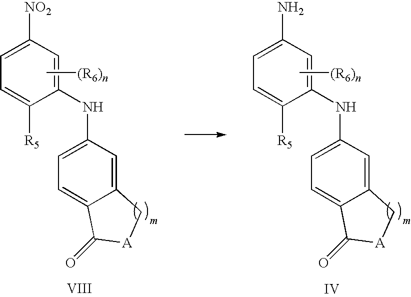

wherein A, R₅, R₆, m and n have the meaning described above. This reaction can be carried out in the presence of a reducing agent such as tin (II) chloride or iron, in a suitable solvent such as ethanol or acetic acid, or alternatively in the presence of hydrogen gas and a palladium catalyst, such as palladium on active carbon, in a suitable solvent such as methyl alcohol, ethyl alcohol or ethyl acetate.

Compounds of formula VII and VIII can be obtained by reacting a compound of formula IX with an amine of formula X, as shown in the following scheme:

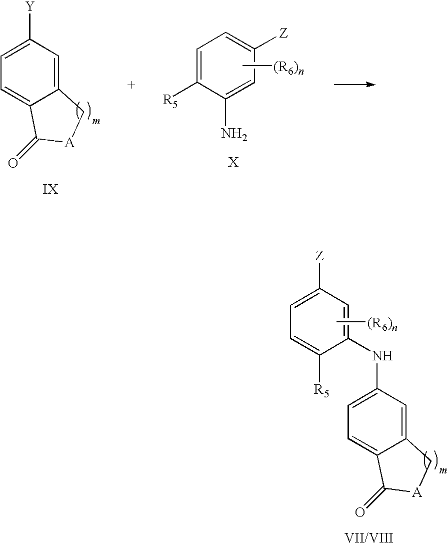

wherein Y represents halogen, preferably bromo, or trifluoromethanesulfonate, Z represents COOR or NO₂, and A, R, R₅, R₆, m and n have the meaning described above. This reaction can be carried out in the presence of a base, such as Cs₂CO₃ or sodium tert-butoxide, in the presence of a palladium catalyst, such as palladium acetate (II) or tris(dibenzylideneacetone)dipalladium(0), and a phosphine such as 2,2′-bis(diphenylphosphino)-1,1′-binaphthyl, in a solvent such as toluene or dioxane.

Alternatively, compounds of formula I wherein R₁₅═H can be obtained by reacting a compound of formula IX with an amine of formula Xa, as shown in the following scheme:

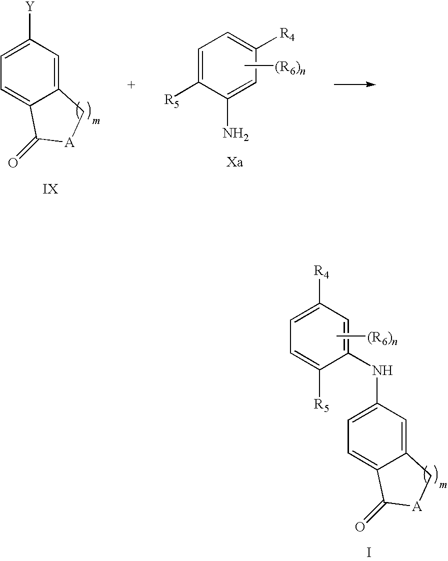

wherein A, R₄, R₅, R₆, m, n and Y have the meaning described above. This reaction is carried out under the same conditions described above for the preparation of compounds VIINIII from compounds I× and X.

The compounds of formula IX wherein A=CR₁R₂ (IXa: A=CR₁R₂, m=1; IXb: A=CR₁R₂, m=2) and Y represents halogen can be obtained by reacting a compound of formula XI with an alkylating agent of formula XII, as shown in the following scheme:

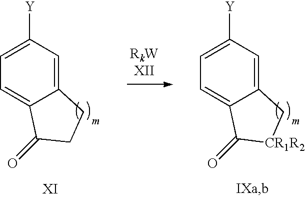

wherein R₁, R₂ and m have the meaning described above, Y represents halogen, preferably bromo, Rₖ represents R₁ or R₂ and W represents halogen or alkylsulfonate, preferably iodo. This reaction can be carried out in the presence of a base such as sodium hydride, in a suitable solvent such as toluene, tetrahydrofuran or dimethylformamide, and at a temperature comprised between room temperature and the temperature of the boiling point of the solvent. When R₁≠R₂, this reaction is carried out in a two-step sequence that involves alkylating a compound of formula XI with an alkylating agent R₁W to give a mono-alkylated intermediate and then reacting this intermediate with a second alkylating agent R₂W to yield the compound of formula IXa,b.

Compounds of formula IX wherein A=NR₃ and m=1 (IXc) can be obtained by reacting a compound of formula XIIIa with an amine of formula XIV, as shown in the following scheme:

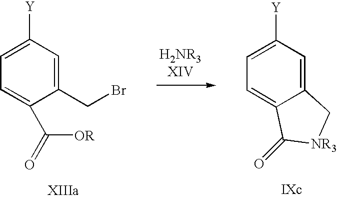

wherein R and R₃ have the meaning described above and Y represents halogen, preferably bromo. This reaction can be carried out in a suitable solvent such as methanol, ethanol or dimethylformamide, optionally in the presence of a base such as a tertiary amine (like triethylamine or N,N-diisopropylethylamine), sodium carbonate or potassium carbonate, and at a temperature comprised between room temperature and the temperature of the boiling point of the solvent. Alternatively, this reaction can be carried out in a two-step sequence that involves bromo displacement from a compound of formula XIIIa by the amine XIV in a suitable solvent such as methanol, ethanol or dimethylformamide, to yield an intermediate aminoester, and final cyclization to the compound of formula IXc by heating in acetic acid or polyphosphoric acid.

Compounds of formula IX wherein Y represents trifluoromethanesulfonate can be obtained starting from a compound of formula XV, as shown in the following scheme:

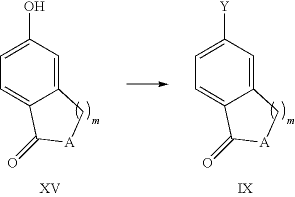

wherein A and m have the meaning described above and Y represents trifluoromethanesulfonate. This reaction can be carried out in the presence of a suitable sulfonylating agent such as trifluoromethanesulfonic anhydride or trifluoromethanesulfonyl chloride, in a suitable solvent such as pyridine or dichloromethane, in the presence of a base such as pyridine or triethylamine, and at a suitable temperature comprised between 0° C. and room temperature.

Compounds of formula XV can be obtained starting from a compound of formula XVI, as shown in the following scheme:

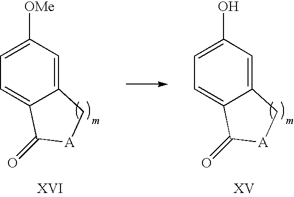

wherein A and m have the meaning described above. This reaction can be carried out in the presence of a strong acid, such as 48% HBr, and at a suitable temperature comprised between room temperature and the temperature of the boiling point of the solvent, or in the presence of a Lewis acid such as boron tribromide, in a suitable solvent such as dichloromethane, and at a temperature comprised preferably between −78° C. and room temperature.

Compounds of formula XVI wherein A=CR₁R₂ (XVIa: A=CR₁R₂, m=1; XVIb: A=CR₁R₂, m=2) can be obtained by reaction of compounds of formula XVII under the same conditions previously described for the conversion of a compound of formula XI into a compound of formula IXa,b, as shown in the following scheme:

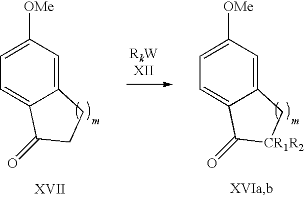

wherein R₁, R₂ and m have the meaning described above.

Compounds of formula XVI wherein A=NR₃ and m=1 (XVIc) can be obtained by reacting a compound of formula XIIIb with an amine of formula XIV, as shown in the following scheme:

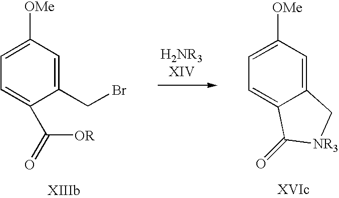

wherein R and R₃ have the meaning described above. This reaction can be carried out under the same reaction conditions described above for the preparation of compounds IXc from XIIIa.

Compounds of formula XIIIa,b can be obtained starting from a compound of formula XVIII, as shown in the following scheme:

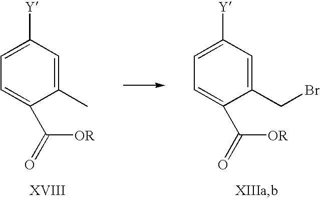

wherein R has the meaning described above and Y′ represents halogen, preferably bromo, or methoxy. This reaction can be carried out in the presence of a suitable halogenating agent, such as N-bromosuccinimide, optionally in the presence of a radical initiator such as 2,2′-azobis(2-methylbutyronitrile) or benzoyl peroxide, in a suitable solvent such as CCl₄, CHCl₃, acetonitrile or chlorobenzene, and at a suitable temperature comprised between room temperature and the temperature of the boiling point of the solvent, optionally irradiating the mixture.

Compounds of formula XVIII can be obtained by reacting a carboxylic acid of formula XIX with an alcohol of formula XX, as shown in the following scheme:

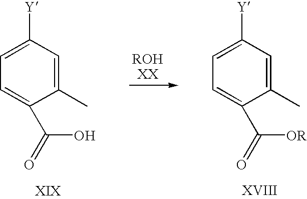

wherein R has the meaning described above and Y′ represents halogen, preferably bromo, or methoxy. This reaction can be carried out in the presence of an inorganic acid such as concentrated sulfuric acid, using the alcohol of formula XX as the solvent, and at a suitable temperature comprised between room temperature and the temperature of the boiling point of the solvent. Alternatively, a compound of formula XIX can be converted into the corresponding acyl chloride by using standard conditions and then the latter can be converted into the corresponding ester of formula XVIII by reaction with an alcohol of formula XX, in the presence of a base such as triethylamine, in a suitable solvent such as for example dichloromethane, and at a suitable temperature comprised between 0° C. and room temperature.

Compounds of formula XVI wherein A=NR₃ (XVIc: m=1; XVId: m=2) can be obtained starting from a compound of formula XXI, as shown in the following scheme:

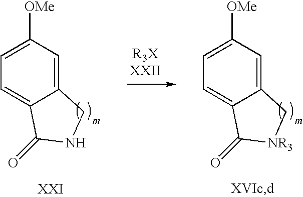

wherein R₃ and m have the meaning described above. When R₃ is an alkyl-type group, this reaction can be carried out by treatment with an alkylating agent such as a halide or alkylsulfonate of formula XXII, preferably an alkyl iodide, in the presence of a base such as sodium hydride, in a suitable solvent such as toluene, tetrahydrofuran or dimethylformamide, and at a temperature comprised between room temperature and the temperature of the boiling point of the solvent. When R₃ is a phenyl or heteroaryl group, this reaction can be carried out by reaction with an halide of formula XXII, preferably a bromide, in the presence of a base, such as K₂CO₃, Na₂CO₃ or K₃PO₄, and a copper catalyst, such as copper(I) iodide, in a solvent such as N-methylpyrrolidone and heating, preferably at reflux.

Alternatively, compounds of formula IX wherein A=NR₃ (IXc: m=1; IXd: m=2) can be obtained in an analogous manner starting from a compound of formula XXIII, as shown in the following scheme:

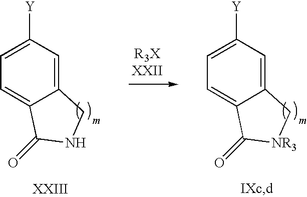

wherein R₃ and m have the meaning described above and Y represents halogen, preferably bromo. This reaction is carried out under the same reaction conditions described above for the preparation of compounds XVIc,d from XXI.

Compounds of formula III, V, VI, X, Xa, XI, XII, XIV, XVII, XIX, XX, XXI, XXII and XXIII are commercially available or can be prepared by methods widely described in the literature, and can be conveniently protected.

Furthermore, some compounds of the present invention can also be obtained from other compounds of formula I by appropriate conversion reactions of functional groups in one or several steps, using well-known reactions in organic chemistry under the reported standard experimental conditions.

Thus, a group R₁₅ ca n be converted into another group R₁₅, resulting in further compounds of formula I. For example, R₁₅═H can be converted into R₁₅═R₁₆ by alkylation with a suitable alkylating agent such as a halide, preferably a iodide, or an alkyl- or arylsulfonate, in the presence of a base such as triethylamine, sodium hydroxide, sodium carbonate, potassium carbonate, sodium hydride or sodium bis(trimethylsilyl)amide, among others, in a suitable solvent such as dichloromethane, chloroform, dimethylformamide, tetrahydrofuran, acetonitrile or toluene, optionally in the presence of a crown ether, and at a temperature comprised between −78° C. and the temperature of the boiling point of the solvent. Likewise, compounds of formula I wherein R₁₅═—COR₁₇ or —SO₂R₁₇ can be obtained from a compound of formula I wherein R₁₅═H by standard procedures, for example by treatment with an acid chloride of formula R₁₇COCl or a sulfonyl chloride of formula R₁₇SO₂Cl and heating.

Other conversions on groups of R₃, R₄ and R₁₅ include, for example:

the conversion of a primary or secondary hydroxyl group into a leaving group, for example an alkylsulfonate or arylsulfonate such as mesylate or tosylate or a halogen such as Cl, Br or I, by reaction with a sulfonyl halide such as methanesulfonyl chloride, in the presence of a base, such as pyridine or triethylamine, in a suitable solvent such as dichloromethane or chloroform, or with a halogenating agent, such as SOCl₂, in a suitable solvent such as tetrahydrofuran, followed by substitution of said leaving group by reaction with an alcohol, amine or thiol, optionally in the presence of a base, such as triethylamine, K₂CO₃, NaH or KOH, and in a suitable solvent such as dimethylformamide, 1,2-dimethoxyethane or acetonitrile;

the conversion of an amine into an amide, carbamate, urea or sulfonamide under standard conditions, for example following the methods disclosed above;

the conversion of an aromatic halide into an aromatic amine by reaction with an amine, optionally in the presence of a suitable solvent, and preferably heating;

the alkylation of an amide by treatment with an alkylating agent under basic conditions.

Some of these interconversion reactions are explained in greater detail in the examples.

As it will be obvious to those skilled in the art, these interconversion reactions can be carried out upon the compounds of formula I as well as upon any suitable synthesis intermediate thereof.

As mentioned previously, the compounds of the present invention act as p38 kinase inhibitors, inducing the reduction of proinflammatory cytokines. Therefore, the compounds of the invention are expected to be useful to treat or prevent diseases in which p38 plays a role in mammals, including human beings. This includes diseases caused by overproduction of cytokines such as TNF-α, IL-1, IL-6 or IL-8. These diseases include, but are not limited to, immune, autoimmune and inflammatory diseases, cardiovascular diseases, infectious diseases, bone resorption disorders, neurodegenerative diseases, proliferative diseases and processes associated with cyclooxygenase-2 induction. Preferred diseases to be treated or prevented with the compounds of the invention are immune, autoimmune and inflammatory diseases.

As an example, immune, autoimmune and inflammatory diseases that can be treated or prevented with the compounds of the present invention include rheumatic diseases (e.g. rheumatoid arthritis, psoriatic arthritis, infectious arthritis, progressive chronic arthritis, deforming arthritis, osteoarthritis, traumatic arthritis, gouty arthritis, Reiter's syndrome, polychondritis, acute synovitis and spondylitis), glomerulonephritis (with or without nephrotic syndrome), autoimmune hematologic disorders (e.g. hemolytic anemia, aplasic anemia, idiopathic thrombocytopenia and neutropenia), autoimmune gastritis and autoimmune inflammatory bowel diseases (e.g. ulcerative colitis and Crohn's disease), host versus graft disease, allograft rejection, chronic thyroiditis, Graves' disease, schleroderma, diabetes (type I and type II), active hepatitis (acute and chronic), primary biliary cirrhosis, myasthenia gravis, multiple sclerosis, systemic lupus erythematosus, psoriasis, atopic dermatitis, contact dermatitis, eczema, skin sunburns, chronic renal insufficiency, Stevens-Johnson syndrome, idiopathic sprue, sarcoidosis, Guillain-Barré syndrome, uveitis, conjunctivitis, keratoconjunctivitis, otitis media, periodontal disease, pulmonary interstitial fibrosis, asthma, bronchitis, rhinitis, sinusitis, pneumoconiosis, pulmonary insufficiency syndrome, pulmonary emphysema, pulmonary fibrosis, silicosis, chronic inflammatory pulmonary disease (e.g. chronic obstructive pulmonary disease) and other inflammatory or obstructive diseases of the airways.

Cardiovascular diseases that can be treated or prevented include, among others, myocardial infarction, cardiac hypertrophy, cardiac insufficiency, ischaemia-reperfusion disorders, thrombosis, thrombin-induced platelet aggregation, acute coronary syndromes, atherosclerosis and cerebrovascular accidents.

Infectious diseases that can be treated or prevented include, among others, sepsis, septic shock, endotoxic shock, sepsis by Gram-negative bacteria, shigellosis, meningitis, cerebral malaria, pneumonia, tuberculosis, viral myocarditis, viral hepatitis (hepatitis A, hepatitis B and hepatitis C), HIV infection, retinitis caused by cytomegalovirus, influenza, herpes, treatment of infections associated with severe burns, myalgias caused by infections, cachexia secondary to infections, and veterinary viral infections such as lentivirus, caprine arthritic virus, visna-maedi virus, feline immunodeficiency virus, bovine immunodeficiency virus or canine immunodeficiency virus.

Bone resorption disorders that can be treated or prevented include osteoporosis, osteoarthritis, traumatic arthritis and gouty arthritis, as well as bone disorders related with multiple myeloma, bone fracture and bone grafting and, in general, all these processes wherein it is necessary to induce osteoblastic activity and increase bone mass.

Neurodegenerative diseases that can be treated or prevented include Alzheimer's disease, Parkinson's disease, cerebral ischaemia and traumatic neurodegenerative disease, among others.

Proliferative diseases that can be treated or prevented include endometriosis, solid tumors, acute and chronic myeloid leukemia, Kaposi sarcoma, multiple myeloma, metastatic melanoma and angiogenic disorders such as ocular neovascularisation and infantile haemangioma.

p38 kinase inhibitors also inhibit the expression of proinflammatory proteins such as cyclooxygenase-2 (COX-2), the enzyme responsible for prostaglandin production. Therefore, the compounds of the present invention can also be used to treat or prevent diseases mediated by COX-2 and especially to treat processes with edema, fever and neuromuscular pain such as cephalea, pain caused by cancer, tooth pain, arthritic pain, hyperalgesia and allodynia.

In vitro and in vivo assays to determine the ability of a compound to inhibit p38 activity are well known in the art. For example, a compound to be tested can be contacted with the purified p38 enzyme to determine whether inhibition of p38 activity occurs. Alternatively, cell-based assays can be used to measure the ability of a compound to inhibit the production of cytokines such as TNFalpha, e.g. in stimulated peripheral blood mononuclear cells (PBMCs) or other cell types. Detailed disclosure of an assay that can be used to test the biological activity of the compounds of the invention as p38 inhibitors can be found below (see Example 22).

For selecting active compounds, testing at 10 μM must result in an activity of more than 50% inhibition in the test provided in Example 22. More preferably, compounds should exhibit more than 50% inhibition at 1 μM, and still more preferably, they should exhibit more than 50% inhibition at 0.1 μM.

The present invention also relates to a pharmaceutical composition which comprises a compound of the present invention (or a pharmaceutically acceptable salt or solvate thereof) and one or more pharmaceutically acceptable excipients. The excipients must be “acceptable” in the sense of being compatible with the other ingredients of the composition and not deleterious to the recipients thereof.

The compounds of the present invention can be administered in the form of any pharmaceutical formulation, the nature of which, as it is well known, will depend upon the nature of the active compound and its route of administration. Any route of administration may be used, for example oral, parenteral, nasal, ocular, rectal and topical administration.

Solid compositions for oral administration include tablets, granulates and capsules. In any case the manufacturing method is based on a simple mixture, dry granulation or wet granulation of the active compound with excipients. These excipients can be, for example, diluents such as lactose, microcrystalline cellulose, mannitol or calcium hydrogenphosphate; binding agents such as for example starch, gelatin or povidone; disintegrants such as sodium carboxymethyl starch or sodium croscarmellose; and lubricating agents such as for example magnesium stearate, stearic acid or talc. Tablets can be additionally coated with suitable excipients by using known techniques with the purpose of delaying their disintegration and absorption in the gastrointestinal tract and thereby provide a sustained action over a longer period, or simply to improve their organoleptic properties or their stability. The active compound can also be incorporated by coating onto inert pellets using natural or synthetic film-coating agents. Soft gelatin capsules are also possible, in which the active compound is mixed with water or an oily medium, for example coconut oil, mineral oil or olive oil.

Powders and granulates for the preparation of oral suspensions by the addition of water can be obtained by mixing the active compound with dispersing or wetting agents; suspending agents and preservatives. Other excipients can also be added, for example sweetening, flavouring and colouring agents.

Liquid forms for oral administration include emulsions, solutions, suspensions, syrups and elixirs containing commonly-used inert diluents, such as purified water, ethanol, sorbitol, glycerol, polyethylene glycols (macrogols) and propylene glycol. Said compositions can also contain coadjuvants such as wetting, suspending, sweetening, flavouring agents, preservatives and buffers.

Injectable preparations, according to the present invention, for parenteral administration, comprise sterile solutions, suspensions or emulsions, in an aqueous or non-aqueous solvent such as propylene glycol, polyethylene glycol or vegetable oils. These compositions can also contain coadjuvants, such as wetting, emulsifying, dispersing agents and preservatives. They may be sterilized by any known method or prepared as sterile solid compositions which will be dissolved in water or any other sterile injectable medium immediately before use. It is also possible to start from sterile materials and keep them under these conditions throughout all the manufacturing process.

For the rectal administration, the active compound can be preferably formulated as a suppository on an oily base, such as for example vegetable oils or solid semisynthetic glycerides, or on a hydrophilic base such as polyethylene glycols (macrogol).

The compounds of the invention can also be formulated for their topical application for the treatment of pathologies occurring in zones or organs accessible through this route, such as eyes, skin and the intestinal tract. Formulations include creams, lotions, gels, powders, solutions and patches wherein the compound is dispersed or dissolved in suitable excipients.

For the nasal administration or for inhalation, the compound can be formulated as an aerosol and it can be conveniently released using suitable propellants.

The dosage and frequency of doses will depend upon the nature and severity of the disease to be treated, the age, the general condition and body weight of the patient, as well as the particular compound administered and the route of administration, among other factors. A representative example of a suitable dosage range is from about 0.01 mg/Kg to about 100 mg/Kg per day, which can be administered as a single or divided doses.

The invention is illustrated by the following examples.

### EXAMPLES

The following abbreviations have been used in the examples:

ACN: acetonitrile
DMF: dimethylformamide
EDC.HCl: N-(3-dimethylaminopropyl)-N′-ethylcarbodiimide hydrochloride

EtOAc: ethyl acetate

EtOH: ethanol
HOBT: 1-hydroxybenzotriazole hydrate
MeOH: methanol
PyBOP: (Benzotriazol-1-yloxy)tripyrrolidinophosphonium hexafluorophosphate

TEA: triethylamine

THF: tetrahydrofuran
t_{R}: retention time
LC-MS: liquid chromatography-mass spectrometry

LC-MS spectra have been performed using the following chromatographic methods:

Method 1: Column Tracer Excel 120, ODSB 5 μm (10 mm×0.21 mm), temperature: 30° C., flow: 0.35 mL/min, eluent: A=ACN, B=0.1% HCOOH, gradient: 0 min 10% A-10 min 90% A-15 min 90% A.

Method 2: Column X-Terra MS C18 5 μm (150 mm×2.1 mm), temperature: 30° C., flow: 0.35 mL/min, eluent: A=ACN, B=10 mM Ammonium bicarbonate, gradient: 0 min 10% A-10 min 90% A-15 min 90% A.

Method 3: Column X-Terra MS C18 5 μm (100 mm×2.1 mm), temperature: 30° C., flow: 0.35 mL/min, eluent: A=ACN, B=0.1% HCOOH, gradient: 0 min 10% A-10 min 90% A-15 min 90% A.

Method 4: Column X-Terra MS C18 5 μm (100 mm×2.1 mm), temperature: 30° C., flow: 0.35 mL/min, eluent: A=ACN, B=10 mM Ammonium bicarbonate, gradient: 0 min 10% A-10 min 90% A-15 min 90% A.

The MS spectra have been obtained with positive electrospray ionization mode over a scan range from 100 to 800 amu.

Preparative HPLC have been performed using the following chromatographic conditions:

Column X-Terra Prep MS C18 5 μm (100 mm×19 mm), flow: 20 mL/min, eluent: A=ACN, B=75 mM Ammonium bicarbonate, gradient.

### Reference Example 1

### Methyl 4-bromo-2-methylbenzoate

To a solution of 4-bromo-2-methylbenzoic acid (6.17 g, 0.29 mol) in MeOH (170 mL), H₂SO₄ 95% (3 mL) was added. It was heated to reflux overnight and allowed to cool to room temperature. The solvent was evaporated and EtOAc was added. The organic phase was washed with saturated NaHCO₃, aq Na₂CO₃ and water. The combined organic phases were dried over Na₂SO₄ and the solvent was evaporated, to afford 6.43 g of the title compound as an oil (yield: 98%).

¹H NMR (300 MHz, CDCl₃) δ (TMS): 2.58 (s, 3H), 3.89 (s, 3H), 7.36 (d, J=1.8 Hz, 1H), 7.41 (dd, J=8.1 Hz, J′=1.8 Hz, 1H), 7.78 (d, J=8.1 Hz, 1H).

### Reference Example 2

### Methyl 4-bromo-2-(bromomethyl)benzoate

To a solution of methyl 4-bromo-2-methylbenzoate (9.60 g, 0.42 mol, obtained in reference example 1) in CCl₄ (150 mL), N-bromosuccinimide (7.46 g, 0.42 mol) and benzoyl peroxide (0.19 g, 0.79 mmol) were added. The reaction mixture was stirred 4 h at room temperature while irradiated with a 250 Watt lamp and it was then filtered to remove the precipitated solids. The filtrate was washed with 1N NaOH and water and it was dried over Na₂SO₄. The solvent was evaporated to afford 11.87 g of the desired compound as an oil that solidified on standing (yield: 92%, uncorrected).

¹H NMR (300 MHz, CDCl₃) 8 (TMS): 3.94 (s, 3H), 4.90 (s, 2H), 7.51 (dd, J=8.4 Hz, J′=2.1 Hz, 1H), 7.63 (d, J=1.8 Hz, 1H), 7.84 (d, J=8.4 Hz, 1H).

### Reference Example 3

### 5-Bromo-2-phenyl-2,3-dihydroisoindol-1-one

To a solution of methyl 4-bromo-2-(bromomethyl)benzoate (4.9 mmol, obtained in reference example 2) in MeOH (40 mL), aniline (0.93 g, 5.1 mmol) and TEA (1.05 mL, 7.6 mmol) were added. The mixture was heated to reflux for 24 h and then allowed to cool to room temperature. The solvent was evaporated and the crude product obtained was purified by chromatography on silica gel using hexane-EtOAc mixtures of increasing polarity as eluent, to afford 1.07 g of the desired compound, impurified with starting aniline. The product was dissolved in CHCl₃ and the organic phase was washed with 1N HCl, dried over Na₂SO₄ and the solvent evaporated to afford 0.98 g of the title compound (yield: 67%).

¹H NMR (300 MHz, CDCl₃) 8 (TMS): 4.85 (s, 2H), 7.18 (m, 1H), 7.46 (m, 2H), 7.64-7.86 (complex signal, 5H)

### Reference Example 3A

### 5-Bromo-2-ethyl-2,3-dihydroisoindol-1-one

To a solution of methyl 4-bromo-2-(bromomethyl)benzoate (1.2 mmol, obtained in reference example 2) in MeOH (10 mL), ethylamine (1.2 mL of a 2M solution in MeOH, 2.4 mmol) was added. The mixture was heated to reflux for 24 h and then allowed to cool to room temperature. The solvent was evaporated and the crude product obtained was purified by chromatography on silica gel using hexane-EtOAc mixtures of increasing polarity as eluent, to afford 0.2 g of the title compound (yield: 72%).

LC-MS (method 1): t_{R}=6.83 min; m/z=240.0/242.0 [M+H]⁺.

### Reference Example 3B

### 5-Bromo-2-(3-hydroxypropyl)-2,3-dihydroisoindol-1-one

Following a similar procedure to that described in reference example 3, but starting from reference example 2 and 3-amino-1-propanol, the desired compound was obtained.

LC-MS (method 1): t_{R}=5.23 min; m/z=270.0/272.0 [M+H]⁺.

### Reference Example 3C

### 5-Bromo-2-cyclopentyl-2,3-dihydroisoindol-1-one

Following a similar procedure to that described in reference example 3A, but starting from reference example 2 and cyclopentylamine, the desired compound was obtained.

LC-MS (method 3): t_{R}=7.62 min; m/z=280.4/282.4 [M+H]⁺.

### Reference Example 3D

### 5-Bromo-2-(2-hydroxyethyl)-2,3-dihydroisoindol-1-one

Following a similar procedure to that described in reference example 3A, but starting from reference example 2 and ethanolamine, the desired compound was obtained.

LC-MS (method 4): t_{R}=4.47 min; m/z=256.3/258.3 [M+H]⁺.

### Reference Example 4

### 5-Bromo-2,2-dimethylindan-1-one

To a suspension of sodium hydride (55% in mineral oil, 1.37 g, 31.3 mmol) in toluene (8.5 mL), 5-bromo-1-indanone (3.00 g, 14.2 mmol) and methyl iodide (4.43 g, 31.3 mmol) were added. The mixture was heated at 90° C. overnight and allowed to cool to room temperature. After adding some drops of MeOH to destroy the excess of hydride, EtOAc and water were added. The phases were separated and the aqueous phase was reextracted twice with EtOAc. The combined organic phases were dried over Na₂SO₄ and the solvent was evaporated. The crude product obtained was purified by chromatography on silica gel using hexane-EtOAc mixtures of increasing polarity as eluent, to afford 2.43 g of the title compound (yield: 72%).

¹H NMR (300 MHz, CDCl₃) 5 (TMS): 1.25 (s, 6H), 2.98 (s, 2H), 7.51 (d, J=8.4 Hz, 1H), 7.60-7.63 (complex signal, 2H).

### Reference Example 5

### 2,2-Dimethyl-6-methoxy-1,2,3,4-tetrahydronaphthalen-1-one

To a suspension of sodium hydride (55% in mineral oil, 26.80 g, 0.55 mol) in benzene (159 mL), 6-methoxy-1,2,3,4-tetrahydronaphthalen-1-one (50.00 g, 0.28 mol) and methyl iodide (99.10 g, 0.69 mol) were added. The mixture was heated to reflux overnight and allowed to cool to room temperature. After adding some drops of MeOH to destroy the excess of hydride, EtOAc and water were added. The phases were separated and the aqueous phase was reextracted with EtOAc. The combined organic phases were dried over Na₂SO₄ and the solvent was evaporated to afford the title compound (quantitative yield).

¹H NMR (80 MHz, CDCl₃) 6 (TMS): 1.19 (s, 6H), 1.94 (t, J=6.5 Hz, 2H), 2.93 (t, J=6.5 Hz, 2H), 3.82 (s, 3H), 6.67 (broad s, 1H), 6.80 (dd, J=9 Hz, J′=2 Hz, 1H), 7.99 (d, J=9 Hz, 1H).

### Reference Example 6

### 2,2-Dimethyl-6-hydroxy-1,2,3,4-tetrahydronaphthalen-1-one

A mixture of 2,2-dimethyl-6-methoxy-1,2,3,4-tetrahydronaphthalen-1-one (20.0 g, 98 mmol, obtained in reference example 5) and 48% aq HBr (279 mL) was heated to reflux for 2 h. Then HBr was distilled off and the reaction crude was allowed to cool to room temperature and diluted with water and ethyl ether. The phases were separated and the product was extracted from the organic phase with 1N NaOH. The basic aqueous phase was acidified with 2N HCl and the solid thus obtained was isolated by filtration and dried under vacuum, to afford 16.06 g of the desired compound as a tan solid (yield: 86%).

¹H NMR (300 MHz, CDCl₃) δ (TMS): 1.21 (s, 6H), 1.96 (t, J=6.3 Hz, 2H), 2.92 (t, J=6.3 Hz, 2H), 5.62 (s, 1H, OH), 6.65 (d, J=2.4 Hz, 1H), 6.76 (dd, J=8.4 Hz, J′=2.4 Hz, 1H), 7.98 (d, J=8.4 Hz, 1H).

### Reference Example 7

### 2,2-Dimethyl-1-oxo-1,2,3,4-tetrahydronaphthalen-6-yl trifluoromethanesulfonate

To a solution of 2,2-dimethyl-6-hydroxy-1,2,3,4-tetrahydronaphthalen-1-one (15.00 g, 78.8 mmol, obtained in reference example 6) in pyridine (40 mL), cooled at 0° C., trifluoromethanesulfonic anhydride (24.46 g, 86.7 mmol) was added. The reaction mixture was allowed to warm to room temperature and stirred overnight. After dilution with water and EtOAc, the phases were separated and the aqueous phase was reextracted 3 times with EtOAc. The combined organic phases were washed with water and twice with 10% HCl, dried over Na₂SO₄ and the solvent was evaporated. The crude product obtained was purified by chromatography on silica gel using hexane-EtOAc mixtures of increasing polarity as eluent, to afford 21.54 g of the desired compound (yield: 85%).

¹H NMR (300 MHz, CDCl₃) δ (TMS): 1.23 (s, 6H), 2.02 (t, J=6.3 Hz, 2H), 3.03 (t, J=6.3 Hz, 2H), 7.15 (d, J=2.4 Hz, 1H), 7.20 (dd, J=8.7 Hz, J′=2.4 Hz, 1H), 8.13 (d, J=8.7 Hz, 1H).

### Reference Example 8

### Ethyl N-[2-(3-methoxyphenyl)ethyl]carbamate

To a solution of 3-methoxyphenetylamine (25.00 g, 0.17 mol) and TEA (25 mL, 0.18 mol) in CH₂Cl₂ (500 mL), cooled at 0° C., ethyl chloroformate (19.53 g, 0.18 mol) was added dropwise and the reaction mixture was stirred at 0° C. for 1.5 h. Water was then added and the phases were separated. The aqueous phase was reextracted with CH₂Cl₂. The combined organic phases were dried over Na₂SO₄ and the solvent was evaporated to afford the desired compound (quantitative yield).

¹H NMR (300 MHz, CDCl₃) 8 (TMS): 1.23 (t, J=7.2 Hz, 3H), 2.78 (t, J=6.9 Hz, 2H), 3.43 (q, J=6.6 Hz, 2H), 3.80 (s, 3H), 4.10 (q, J=6.9 Hz, 2H), 4.69 (broad s, 1H), 6.74-6.79 (complex signal, 3H), 7.22 (t, J=7.8 Hz, 1H).

### Reference Example 9

### 6-Methoxy-1,2,3,4-tetrahydroisoquinolin-1-one

A mixture of ethyl N-[2-(3-methoxyphenyl)ethyl]carbamate (18.98 g, 85.0 mmol, obtained in reference example 8) and polyphosphoric acid (60 g) was heated at 120° C. for 3 h and then allowed to cool to 60° C. Water and EtOAc were added and the mixture was allowed to cool to room temperature. The phases were separated and the aqueous phase was reextracted several times with CHCl₃. The combined organic phases were dried over Na₂SO₄ and the solvent was evaporated. The crude product obtained was purified by chromatography on silica gel using EtOAc-MeOH mixtures of increasing polarity as eluent, to afford 10.24 g of the desired compound (yield: 68%).

¹H NMR (300 MHz, CDCl₃) 8 (TMS): 2.97 (m, 2H), 3.55 (m, 2H), 3.85 (s, 3H), 6.31 (broad s, 1H), 6.70 (d, J=2.1 Hz, 1H), 6.85 (dd, J=8.7 Hz, J′=2.4 Hz, 1H), 8.01 (d, J=8.4 Hz, 1H).

### Reference Example 10

### 2-(2-Chlorophenyl)-6-methoxy-1,2,3,4-tetrahydroisoquinolin-1-one

To a solution of 6-methoxy-1,2,3,4-tetrahydroisoquinolin-1-one (1.50 g, 8.5 mmol, obtained in reference example 9) in N-methylpyrrolidone (4 mL) under argon, 1-bromo-2-chlorobenzene (2.34 g, 12.3 mmol), copper (I) iodide (0.33 g, 1.7 mmol) and potassium carbonate (2.33 g, 16.9 mmol) were added and the mixture was heated at 200° C. overnight. It was allowed to cool and CHCl₃ and 1N NaOH were added. The phases were separated and the aqueous phase was reextracted 2 times with CHCl₃. The combined organic phases were dried over Na₂SO₄ and the solvent was evaporated. The crude product thus obtained was purified by chromatography on silica gel using hexane-EtOAc mixtures of increasing polarity as eluent, to afford 2.01 g of the desired compound (yield: 77%).

LC-MS (method 1): t_{R}=8.05 min; m/z=288.1/290.1 [M+H]⁺.

### Reference Example 11

### 2-(2-Chlorophenyl)-6-hydroxy-1,2,3,4-tetrahydroisoquinolin-1-one

To a solution of 2-(2-chlorophenyl)-6-methoxy-1,2,3,4-tetrahydroisoquinolin-1-one (2.01 g, 7.0 mmol, obtained in reference example 10) in dry CH₂Cl₂ (40 mL) under argon, cooled at −78° C., boron tribromide (1M in CH₂Cl₂, 13.9 mL, 13.9 mmol) was added. The mixture was allowed to warm to room temperature and stirred overnight. After cooling with ice, 1N HCl was added and the mixture was stirred at 30° C. for 30 min. The phases were then separated and the aqueous phase was reextracted with CHCl₃. The combined organic phases were dried over Na₂SO₄ and the solvent was evaporated to afford 1.86 g of the desired compound (yield: 98%).

LC-MS (method 1): t_{R}=6.41 min; m/z=274.1/276.1 [M+H]⁺.

### Reference Example 12

### 2-(2-Chlorophenyl)-1-oxo-1,2,3,4-tetrahydroisoquinolin-6-yl trifluoromethanesulfonate

To a solution of 2-(2-chlorophenyl)-6-hydroxy-1,2,3,4-tetrahydroisoquinolin-1-one (1.82 g, 6.7 mmol, obtained in reference example 11) in CH₂Cl₂ (50 mL), pyridine (1.1 mL, 13.3 mmol) was added. The solution was cooled at 0° C. and trifluoromethanesulfonic anhydride (2.06 g, 7.3 mmol) was added. The reaction mixture was allowed to warm to room temperature and stirred overnight. After dilution with water, the phases were separated and the aqueous phase was reextracted with CH₂Cl₂. The combined organic phases were washed with 1N HCl, dried over Na₂SO₄ and the solvent was evaporated. The crude product obtained was purified by chromatography on silica gel using hexane-EtOAc mixtures of increasing polarity as eluent, to afford 2.14 g of the desired compound (yield: 80%).

LC-MS (method 1): t_{R}=9.65 min; m/z=406.0/408.0 [M+H]⁺.

### Reference Example 13

### 5-(2-Methyl-5-nitrophenylamino)-2-phenyl-2,3-dihydroisoindol-1-one

A solution of 5-bromo-2-phenyl-2,3-dihydroisoindol-1-one (200 mg, 0.69 mmol, obtained in reference example 3) in toluene (17 mL) was refluxed for 30 min under argon and then allowed to cool to room temperature. Palladium acetate (II) (12 mg, 0.05 mmol), (±) 2,2′-bis(diphenylphosphino)-1,1′-binaphthyl (32 mg, 0.05 mmol), potassium tert-butoxide (110 mg, 0.98 mmol) and 2-methyl-5-nitroaniline (126 mg, 0.83 mmol) were added. The mixture was inertized with argon and it was heated at 90° C. overnight. The reaction mixture was allowed to cool to room temperature and CHCl₃ and water were added. The phases were separated and the aqueous phase was reextracted with CHCl₃. The combined organic phases were washed with 3N HCl and 1N NaOH and dried over Na₂SO₄. The solvent was evaporated and the crude product thus obtained was purified by chromatography on silica gel using hexane-EtOAc mixtures of increasing polarity as eluent, to afford 200 mg of the title compound (yield: 80%).

LC-MS (method 1): t_{R}=9.91 min; m/z=358.0 [M−H]⁻.

### Reference Examples 14-21

Following a similar procedure to that described in reference example 13, but starting from the appropriate compounds in each case, the compounds in the following table were obtained:

| | | | LC-MS | | |
|---|---|---|---|---|---|
| Reference | | | | t_{R} | m/z |
| example | Compound name | Starting products | Method | (min) | [M + H]⁺ |
| 14 | Methyl 4-methyl-3-(1-oxo-2- | Reference example 3 | 1 | 9.43 | 373.3 |
| | phenyl-2,3-dihydroisoindol-5- | and methyl 3-amino- | | | |
| | ylamino)benzoate | 4-methylbenzoate | | | |
| 15 | Methyl 3-(2-ethyl-1-oxo-2,3- | Reference example | 1 | 7.97 | 325.1 |
| | dihydroisoindol-5-ylamino)-4- | 3A and methyl 3- | | | |
| | methylbenzoate | amino-4- | | | |
| | | methylbenzoate | | | |
| 15A | Methyl 3-(2-(3-hydroxypropyl)- | Reference example | 1 | 6.90 | 353.0 [M − H]⁻ |
| | 1-oxo-2,3-dihydroisoindol-5- | 3B and methyl 3- | | | |
| | ylamino)-4-methylbenzoate | amino-4- | | | |
| | | methylbenzoate | | | |
| 15B | 2-(3-Hydroxypropyl)-5-(2- | Reference example | 1 | 7.02 | 342.1 |
| | methyl-5-nitrophenylamino)- | 3B and 2-methyl-5- | | | |
| | 2,3-dihydroisoindol-1-one | nitroaniline | | | |
| 15C | Methyl 3-(2-(2-hydroxyethyl)- | Reference example | 4 | 5.67 | 341.4 |
| | 1-oxo-2,3-dihydroisoindol-5- | 3D and methyl 3- | | | |
| | ylamino)-4-methylbenzoate | amino-4- | | | |
| | | methylbenzoate | | | |
| | | (Cs₂CO₃ was used | | | |
| | | instead of potassium | | | |
| | | tert-butoxide) | | | |
| 16 | 2,2-Dimethyl-5-(2-methyl-5- | Reference example 4 | 1 | 9.47 | 311.2 |
| | nitrophenylamino)indan-1-one | and 2-methyl-5- | | | |
| | | nitroaniline | | | |
| 17 | Methyl 3-(2,2-dimethyl-1- | Reference example 4 | 1 | 9.05 | 324.2 |
| | oxoindan-5-ylamino)-4- | and methyl 3-amino- | | | |
| | methylbenzoate | 4-methylbenzoate | | | |
| 17A | Ethyl 3-(2,2-dimethyl-1- | Reference example 4 | 1 | 9.78 | 324.0 |
| | oxoindan-5-ylamino)benzoate | and ethyl 3- | | | |
| | | aminobenzoate | | | |
| 18 | 2,2-Dimethyl-5-(3- | Reference example 4 | 1 | 8.93 | 297.2 |
| | nitrophenylamino)indan-1-one | and 3-nitroaniline | | | |
| 19 | 2,2-Dimethyl-5-(4-methyl-3- | Reference example 4 | 1 | 9.29 | 311.3 |
| | nitrophenylamino)indan-1-one | and 4-methyl-3- | | | |
| | | nitroaniline | | | |
| 20 | 2,2-Dimethyl-6-(2-methyl-5- | Reference example 7 | 2 | 10.47 | 325.3 |
| | nitrophenylamino)-1,2,3,4- | and 2-methyl-5- | | | |
| | tetrahydronaphthalen-1-one | nitroaniline | | | |
| 21 | Methyl 3-(2,2-dimethyl-1-oxo- | Reference example 7 | 2 | 10.46 | 338.3 |
| | 1,2,3,4-tetrahydro- | and methyl 3-amino- | | | |
| | naphthalen-6-ylamino)-4- | 4-methylbenzoate | | | |
| | methylbenzoate | | | | |

### Reference Example 22

### 4-Methyl-3-(1-oxo-2-phenyl-2,3-dihydroisoindol-5-ylamino)benzoic acid

To a solution of methyl 4-methyl-3-(1-oxo-2-phenyl-2,3-dihydroisoindol-5-ylamino)benzoate (0.7 g, 1.9 mmol, obtained in reference example 14) in EtOH (39 mL), a solution of KOH (1 g, 18.8 mmol) in water (3 mL) was added and the mixture was heated to reflux for 2 h. After cooling to room temperature, the solvent was evaporated and the residue was diluted with water. The solution was acidified with 6N HCl and extracted with CHCl₃. The organic phase was dried over Na₂SO₄ and the solvent was evaporated to afford the title compound (quantitative yield). LC-MS (method 1): t_{R}=8.31 min; m/z=359.2 [M+H]⁺.

### Reference Examples 23-25

Following a similar procedure to that described in reference example 22, but starting from the appropriate compounds in each case, the compounds in the following table were obtained:

| | | | LC-MS | | |
|---|---|---|---|---|---|
| Reference | | | | t_{R} | m/z |
| example | Compound name | Starting products | Method | (min) | [M + H]⁺ |
| 23 | 3-(2-Ethyl-1-oxo-2,3- | Reference example | 1 | — | NMR |
| | dihydroisoindol-5-ylamino)-4- | 15 | | | (see |
| | methylbenzoic acid | | | | below) |
| 23A | 3-[2-(3-Hydroxypropyl)-1-oxo- | Reference example | 1 | 5.78 | 341.2 |
| | 2,3-dihydroisoindol-5- | 15A | | | |
| | ylamino]-4-methylbenzoic acid | | | | |
| 23B | 3-[2-(2-Hydroxyethyl)-1-oxo- | Reference example | 4 | 3.05 | 327.4 |
| | 2,3-dihydroisoindol-5- | 15C | | | |
| | ylamino]-4-methylbenzoic acid | | | | |
| 24 | 3-(2,2-Dimethyl-1-oxoindan-5- | Reference example | 1 | 7.40 | 310.3 |
| | ylamino)-4-methylbenzoic | 17 | | | |
| | acid | | | | |
| 24A | 3-(2,2-Dimethyl-1-oxoindan-5- | Reference example | 1 | 7.48 | 294.1 [M − H]⁻ |
| | ylamino)benzoic acid | 17A | | | |
| 25 | 3-(2,2-Dimethyl-1-oxo-1,2,3,4- | Reference example | 2 | 5.91 | 324.3 |
| | tetrahydronaphthalen-6- | 21 | | | |
| | ylamino)-4-methylbenzoic | | | | |
| | acid | | | | |

Reference example 23: ¹H NMR (300 MHz, CDCl₃) 8 (TMS): 1.25 (t, J=7.2 Hz, 3H), 2.33 (s, 3H), 3.64 (q, J=7.2 Hz, 2H), 4.30 (s, 2H), 5.68 (broad s, 1H), 6.90-6.95 (complex signal, 2H), 7.34 (d, J=8.1 Hz, 1H), 7.71 (d, J=8.1 Hz, 1H), 7.75 (dd, J=7.8 Hz, J′=1.8 Hz, 1H), 7.99 (d, J=1.5 Hz, 1H).

### Reference Example 26

### 5-(5-Amino-2-methylphenylamino)-2-phenyl-2,3-dihydroisoindol-1-one

To a solution of 5-(2-methyl-5-nitrophenylamino)-2-phenyl-2,3-dihydroisoindol-1-one (0.25 g, 0.69 mmol, obtained in reference example 13) in EtOH (16 mL), tin (II) chloride (0.64 g, 3.45 mmol) was added and the mixture was heated to reflux for 3 h. It was allowed to cool and diluted with CHCl₃. The organic phase was washed with saturated NaHCO₃ and brine, and dried over Na₂SO₄. The solvent was evaporated and the crude product thus obtained was purified by chromatography on silica gel using hexane-EtOAc mixtures of increasing polarity as eluent, to afford 0.14 g of the title compound (yield: 61%).

LC-MS (method 1): t_{R}=6.32 min; m/z=330.1 [M+H]⁺.

### Reference Example 26A

### 5-(5-Amino-2-methylphenylamino)-2-(3-hydroxypropyl)-2,3-dihydroisoindol-1-one

Following a similar procedure to that described in reference example 26, but starting from 2-(3-hydroxypropyl)-5-(2-methyl-5-nitrophenylamino)-2,3-dihydroisoindol-1-one (obtained in reference example 15B), the desired compound was obtained.

LC-MS (method 1): t_{R}=3.97 min; m/z=312.2 [M+H]⁺.

### Reference Examples 27-30

Following a similar procedure to that described in reference example 26, but starting from the appropriate compound in each case, the compounds in the following table were obtained:

| | | | LC-MS | | |
|---|---|---|---|---|---|
| Reference | | | | t_{R} | m/z |
| example | Compound name | Starting products | Method | (min) | [M + H]⁺ |
| 27 | 5-(5-Amino-2- | Reference example | 1 | 5.77 | 281.2 |
| | methylphenylamino)-2,2- | 16 | | | |
| | dimethylindan-1-one | | | | |
| 28 | 5-(3-Aminophenylamino)-2,2- | Reference example | 1 | 5.65 | 267.2 |
| | dimethylindan-1-one | 18 | | | |
| 29 | 5-(3-Amino-4- | Reference example | 1 | 6.93 | 281.3 |
| | methylphenylamino)-2,2- | 19 | | | |
| | dimethylindan-1-one | | | | |
| 30 | 6-(5-Amino-2- | Reference example | 2 | 8.84 | 295.4 |
| | methylphenylamino)-2,2- | 20 | | | |
| | dimethyl-1,2,3,4- | | | | |
| | tetrahydronaphthalen-1-one | | | | |

### Reference Example 31

### 3-Amino-N-cyclopropyl-4-fluorobenzamide

To a solution of 3-amino-4-fluorobenzoic acid (0.30 g, 1.93 mmol) in DMF (27 mL), EDC.HCl (0.41 g, 2.11 mmol), HOBT (0.26 g, 1.93 mmol), and N-methylmorpholine (0.58 g, 5.79 mmol)) were added and the mixture was stirred at room temperature for 1 h. Cyclopropylamine (0.11 g, 1.93 mmol) was added and the mixture was stirred at room temperature overnight. The solvent was evaporated and CHCl₃ and water were added. The phases were separated and the organic phase was washed with saturated NaHCO₃ and dried over Na₂SO₄. The solvent was evaporated and the crude product obtained was purified by chromatography on silica gel using hexane-EtOAc mixtures of increasing polarity as eluent, to afford 0.35 g of the title compound (yield: 92%).

LC-MS (method 1): t_{R}=4.24 min; m/z=195.1 [M+H]⁺.

### Reference Examples 32-33

Following a similar procedure to that described in reference example 31, but starting from the appropriate acid in each case, the compounds in the following table were obtained:

| | | | LC-MS | | |
|---|---|---|---|---|---|
| Reference | | | | t_{R} | m/z |
| example | Compound name | Starting products | Method | (min) | [M + H]⁺ |
| 32 | 3-Amino-N-cyclopropyl-4- | 3-Amino-4- | 1 | 2.77 | 207.1 |
| | methoxybenzamide | methoxybenzoic acid | | | |
| 33 | 3-Amino-4-chloro-N- | 3-Amino-4- | 1 | 5.28 | 211.4/213.4 |
| | cyclopropylbenzamide | chlorobenzoic acid | | | |

### Reference Example 34

### 2-(Pyrrolidin-1-yl)isonicotinic acid

A solution of 2-chloroisonicotinic acid (0.25 g, 1.58 mmol) in pyrrolidine (1.5 mL) was heated at 80° C. overnight. The solvent was evaporated, water and CHCl₃ were added and the phases were separated. The pH of the aqueous phase was adjusted to 5, precipitating a solid that was filtered and washed with water and CHCl₃. After drying the product under vacuum, 95 mg of the title compound were obtained (yield: 31%).

LC-MS (method 1): t_{R}=1.14 min; m/z=193.1 [M+H]⁺.

### Reference Example 35

### 3-Amino-N-cyclopropyl-4-methylbenzamide

Following a similar procedure to that described in reference example 31, but starting from 3-amino-4-methylbenzoic acid and cyclopropylamine, the desired compound was obtained.

LC-MS (method 2): t_{R}=4.44 min; m/z=191.5 [M+H]⁺.

### Example 1

### N-Cyclopropyl-4-methyl-3-(1-oxo-2-phenyl-2,3-dihydroisoindol-5-ylamino)benzamide

To a solution of 4-methyl-3-(1-oxo-2-phenyl-2,3-dihydroisoindol-5-ylamino)benzoic acid (100 mg, 0.28 mmol, obtained in reference example 22) in DMF (4 mL), EDC.HCl (59 mg, 0.31 mmol), HOBT (37 mg, 0.28 mmol), and N-methylmorpholine (0.08 g, 0.84 mmol)) were added and the mixture was stirred at room temperature for 1 h. Cyclopropylamine (15 mg, 0.28 mmol) was added and the mixture was stirred at room temperature overnight. The solvent was evaporated and CHCl₃ and water were added. The phases were separated and the organic phase was washed with saturated NaHCO₃ and dried over Na₂SO₄. The solvent was evaporated and the crude product obtained was purified by chromatography on silica gel using hexane-EtOAc mixtures of increasing polarity as eluent, to afford 96 mg of the title compound (yield: 86%).

LC-MS (method 1): t_{R}=8.35 min; m/z=398.2 [M+H]⁺.

### Examples 1A-1D

Following a similar procedure to that described in example 1, but starting from the appropriate compounds in each case, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 1A | 4,N-Dimethyl-3-(1-oxo-2- | Reference example | 1 | 7.84 | 370.0 [M − H]⁻ |
| | phenyl-2,3-dihydroisoindol-5- | 22 and methylamine | | | |
| | ylamino)benzamide | | | | |
| 1B | N-Cyclopropyl-3-(2-ethyl-1- | Reference example | 1 | 6.58 | 350.2 |
| | oxo-2,3-dihydroisoindol-5- | 23 and | | | |
| | ylamino)-4-methylbenzamide | cyclopropylamine | | | |
| 1C | N-Cyclopropyl-3-[2-(3- | Reference example | 1 | 5.79 | 380.2 |
| | hydroxypropyl)-1-oxo-2,3- | 23A and | | | |
| | dihydroisoindol-5-ylamino]-4- | cyclopropylamine | | | |
| | methylbenzamide | | | | |
| 1D | N-Cyclopropyl-3-[2-(2- | Reference example | 4 | 4.80 | 366.2 |
| | hydroxyethyl)-1-oxo-2,3- | 23B and | | | |
| | dihydroisoindol-5-ylamino]-4- | cyclopropylamine | | | |
| | methylbenzamide | | | | |

### Example 2

### N-Cyclopropyl-3-(2,2-dimethyl-1-oxoindan-5-ylamino)-4-methylbenzamide

Following a similar procedure to that described in example 1, but starting from 3-(2,2-dimethyl-1-oxoindan-5-ylamino)-4-methylbenzoic acid (obtained in reference example 24), the desired compound was obtained.

LC-MS (method 1): t_{R}=7.74 min; m/z=349.3 [M+H]⁺.

### Examples 2A-2G

Following a similar procedure to that described in example 2, but starting from the appropriate amine in each case, the compounds in the following table were obtained:

| | | | LC-MS | | |
|---|---|---|---|---|---|
| | | | | t_{R} | m/z |
| Example | Compound name | Starting amine | Method | (min) | [M + H]⁺ |
| 2A | N-Cyclopropylmethyl-3-(2,2- | Cyclopropylmethylamine | 1 | 8.18 | 363.3 |
| | dimethyl-1-oxoindan-5- | | | | |
| | ylamino)-4-methylbenzamide | | | | |
| 2B | 4,N-Dimethyl-3-(2,2-dimethyl- | Methylamine | 1 | 6.94 | 323.3 |
| | 1-oxoindan-5- | | | | |
| | ylamino)benzamide | | | | |
| 2C | 3-(2,2-Dimethyl-1-oxoindan-5- | Aniline | 1 | 9.01 | 385.3 |
| | ylamino)-4-methyl-N- | | | | |
| | phenylbenzamide | | | | |
| 2D | 3-(2,2-Dimethyl-1-oxoindan-5- | 3-Aminopyridine | 1 | 6.38 | 386.2 |
| | ylamino)-4-methyl-N-(3- | | | | |
| | pyridyl)benzamide | | | | |
| 2E | N-Benzyl-3-(2,2-dimethyl-1- | Benzylamine | 1 | 8.92 | 399.3 |
| | oxoindan-5-ylamino)-4- | | | | |
| | methylbenzamide | | | | |
| 2F | 3-(2,2-Dimethyl-1-oxoindan-5- | 2-Aminothiazole | 1 | 8.71 | 392.2 |
| | ylamino)-4-methyl-N-(2- | | | | |
| | thiazolyl)benzamide | | | | |
| 2G | 3-(2,2-Dimethyl-1-oxoindan-5- | Dimethylamine | 2 | 7.65 | 337.4 |
| | ylamino)-4,N,N-trimethyl | | | | |
| | benzamide | | | | |

### Example 3

### N-Cyclopropyl-3-(2,2-dimethyl-1-oxo-1,2,3,4-tetrahydronaphthalen-6-ylamino)-4-methylbenzamide

Following a similar procedure to that described in example 1, but starting from 3-(2,2-dimethyl-1-oxo-1,2,3,4-tetrahydronaphthalen-6-ylamino)-4-methylbenzoic acid (obtained in reference example 25), the desired compound was obtained.

LC-MS (method 2): t_{R}=8.77 min; m/z=363.3 [M+H]⁺.

### Example 4

### N-[4-methyl-3-(1-oxo-2-phenyl-2,3-dihydroisoindol-5-ylamino)phenyl]furan-3-carboxamide

To a solution of 5-(5-amino-2-methylphenylamino)-2-phenyl-2,3-dihydroisoindol-1-one (70 mg, 0.21 mmol, obtained in reference example 26) in DMF (6 mL), 3-furoic acid (28 mg, 0.25 mmol), HOBT (28 mg, 0.21 mmol), PyBOP (107 mg, 0.21 mmol) and N,N-diisopropylethylamine (0.11 mL) were added and the mixture was stirred at room temperature overnight. The solvent was evaporated and CHCl₃ and saturated NaHCO₃ were added. The phases were separated and the organic phase was dried over Na₂SO₄. The solvent was evaporated and the crude product thus obtained was purified by preparative HPLC, to afford 8 mg of the title compound (yield: 9%).

LC-MS (method 1): t_{R}=9.21 min; m/z=422.0 [M−H]⁻.

### Examples 4A-4B

Following a similar procedure to that described in example 4, but starting from the appropriate compounds in each case, the compounds in the following table were obtained:

| | | | LC-MS | | |
|---|---|---|---|---|---|
| | | | | t_{R} | m/z |
| Example | Compound name | Starting products | Method | (min) | [M + H]⁺ |
| 4A | 2-Cyclopropyl-N-[4-methyl-3- | Reference example | 1 | 9.20 | 412.2 |
| | (1-oxo-2-phenyl-2,3- | 26 and | | | |
| | dihydroisoindol-5- | cyclopropylacetic acid | | | |
| | ylamino)phenyl]acetamide | | | | |
| 4B | 2-Cyclopropyl-N-[3-(2-(3- | Reference example | 1 | 6.57 | 392.1 [M − H]⁻ |
| | hydroxypropyl)-1-oxo-2,3- | 26A and | | | |
| | dihydroisoindol-5-ylamino)-4- | cyclopropylacetic acid | | | |
| | methylphenyl]acetamide | | | | |

### Example 5

### N-[3-(2,2-Dimethyl-1-oxo-1,2,3,4-tetrahydronaphthalen-6-ylamino)-4-methylphenyl]furan-3-carboxamide

Following a similar procedure to that described in example 4, but starting from 6-(5-amino-2-methylphenylamino)-2,2-dimethyl-1,2,3,4-tetrahydronaphthalen-1-one (obtained in reference example 30), the desired compound was obtained.

LC-MS (method 2): t_{R}=9.64 min; m/z=389.3 [M+H]⁺.

### Example 6

### N-[3-(2,2-Dimethyl-1-oxoindan-5-ylamino)-4-methylphenyl]cyclopropylcarboxamide

Following a similar procedure to that described in example 4, but starting from 5-(5-amino-2-methylphenylamino)-2,2-dimethylindan-1-one (obtained in reference example 27) and cyclopropanecarboxylic acid, the desired compound was obtained.

LC-MS (method 1): t_{R}=8.30 min; m/z=349.2 [M+H]⁺.

### Examples 6A-6E

Following a similar procedure to that described in example 6, but starting from the appropriate acid in each case, the compounds in the following table were obtained:

| | | | LC-MS | | |
|---|---|---|---|---|---|
| | | | | t_{R} | m/z |
| Example | Compound name | Starting acid | Method | (min) | [M + H]⁺ |
| 6A | 2-Cyclopropyl-N-[3-(2,2- | Cyclopropylacetic | 1 | 8.33 | 363.3 |
| | dimethyl-1-oxoindan-5- | acid | | | |
| | ylamino)-4- | | | | |
| | methylphenyl]acetamide | | | | |
| 6B | N-[3-(2,2-Dimethyl-1- | 3-Furoic acid | 1 | 8.33 | 375.3 |
| | oxoindan-5-ylamino)-4- | | | | |
| | methylphenyl]furan-3- | | | | |
| | carboxamide | | | | |
| 6C | N-[3-(2,2-Dimethyl-1- | 2- | 1 | 9.12 | 391.2 |
| | oxoindan-5-ylamino)-4- | Thiophenecarboxylic | | | |
| | methylphenyl]thiophene-2- | acid | | | |
| | carboxamide | | | | |
| 6D | 2-Chloro-N-[3-(2,2-dimethyl-1- | 2-Chloroisonicotinic | 1 | 9.17 | 420.2/422.2 |
| | oxoindan-5-ylamino)-4- | acid | | | |
| | methylphenyl]isonicotinamide | | | | |
| 6E | N-[3-(2,2-Dimethyl-1- | Reference example | 1 | 6.16 | 455.4 |
| | oxoindan-5-ylamino)-4- | 34 | | | |
| | methylphenyl]-2-(pyrrolidin-1- | | | | |
| | yl)isonicotinamide | | | | |

### Example 7

### 2-Cyclopropyl-N-[3-(2,2-dimethyl-1-oxo-indan-5-ylamino)phenyl]acetamide

Following a similar procedure to that described in example 4, but starting from 5-(3-aminophenylamino)-2,2-dimethylindan-1-one (obtained in reference example 28) and cyclopropylacetic acid, the desired compound was obtained.

LC-MS (method 1): t_{R}=8.15 min; m/z=349.3 [M+H]⁺.

### Example 8

### N-[3-(2,2-Dimethyl-1-oxoindan-5-ylamino)-4-methylphenyl]acetamide

To a solution of acetyl chloride (28 mg, 0.36 mmol) in CHCl₃ (5 mL), cooled at 0° C., TEA (54 mg, 0.54 mmol) and a solution of 5-(5-amino-2-methylphenylamino)-2,2-dimethylindan-1-one (0.1 g, 0.36 mmol, obtained in reference example 27) in CHCl₃ (5 mL) were added under argon and the mixture was stirred at room temperature overnight. It was then diluted with CHCl₃ and water and the phases were separated. The aqueous phase was reextracted with CHCl₃ and the combined organic phases were washed with brine and dried over Na₂SO₄. The solvent was evaporated and the crude product thus obtained was purified by chromatography on silica gel using hexane-EtOAc mixtures of increasing polarity as eluent, to afford 43 mg of the title compound (yield: 37%).

LC-MS (method 1): t_{R}=7.37 min; m/z=323.3 [M+H]⁺.

### Example 9

### 1-[3-(2,2-Dimethyl-1-oxoindan-5-ylamino)-4-methylphenyl]-3-isopropylurea

To a solution of 5-(5-amino-2-methylphenylamino)-2,2-dimethylindan-1-one (0.10 g, 0.36 mmol, obtained in reference example 27) in DMF (2 mL), isopropyl isocyanate (36 mg, 0.43 mmol) was added under argon and the mixture was heated at 70° C. overnight. The solvent was evaporated and the crude product thus obtained was purified by chromatography on silica gel using hexane-EtOAc mixtures of increasing polarity as eluent, to afford 34 mg of the title compound (yield: 26%).

LC-MS (method 1): t_{R}=8.24 min; m/z=366.1 [M+H]⁺.

### Example 10

### N-Cyclopropyl-3-[N-(2,2-dimethyl-1-oxoindan-5-yl)-N-methylamino]-4-methylbenzamide

To a solution of N-cyclopropyl-3-(2,2-dimethyl-1-oxoindan-5-ylamino)-4-methylbenzamide (0.1 g, 0.29 mmol, obtained in example 2) in dry THF (6 mL) cooled at −78° C., sodium bis(trimethylsilyl)amide (0.29 mL of a 2M solution in THF, 0.58 mmol) was added under argon. The cooling bath was removed and the mixture was stirred at room temperature for 45 min. After cooling again at −78° C., methyl iodide (40 mg, 0.29 mmol) was added. The cooling bath was removed and the reaction mixture was stirred at room temperature for 3 h. Then, 2 mL of saturated NH₄Cl were added and the mixture was diluted with CH₂Cl₂ and water. The phases were separated and the organic phase was dried over Na₂SO₄. The solvent was evaporated and the crude product thus obtained was purified by preparative HPLC, to afford 55 mg of the title compound (yield: 53%).

LC-MS (method 1): t_{R}=8.41 min; m/z=363.1 [M+H]⁺.

### Example 11

### N-Cyclopropyl-3-[N-(2,2-dimethyl-1-oxoindan-5-yl)-N-(3-hydroxypropyl)amino]-4-methylbenzamide

### a) N-Cyclopropyl-3-[N-(2,2-dimethyl-1-oxoindan-5-yl)-N-(3-(tetrahydropyran-2-yloxy)propyl)amino]-4-methylbenzamide

To a suspension of N-cyclopropyl-3-(2,2-dimethyl-1-oxoindan-5-ylamino)-4-methylbenzamide (0.2 g, 0.57 mmol, obtained in example 2) in dry toluene (6.5 mL), sodium hydride (50 mg, 60% dispersion in oil, 1.14 mmol) and 15-crown-5 (4 mg, 0.02 mmol) were added under argon and the mixture was stirred at room temperature for 20 min. Then, 3-bromopropanol tetrahydropyranyl ether (0.13 g, 0.57 mmol) was added and the mixture was heated at 90° C. overnight. It was allowed to cool and diluted with EtOAc and saturated NaHCO₃. The phases were separated and the organic phase was dried over Na₂SO₄. The solvent was evaporated to afford the desired compound (quantitative yield).

LC-MS (method 1): t_{R}=9.74 min; m/z=491.2 [M+H]⁺.

### b) Title Compound

A solution of N-cyclopropyl-3-[N-(2,2-dimethyl-1-oxoindan-5-yl)-N-(3-(tetrahydropyran-2-yloxy)propyl)amino]-4-methylbenzamide (0.57 mmol, obtained in section a) in a mixture of acetic acid (6.5 mL), THF (3.25 mL) and water (1.6 mL) was heated at 50° C. overnight. The solvent was evaporated and the residue was diluted with EtOAc and washed with saturated NaHCO₃. The organic phase was dried over Na₂SO₄, the solvent was evaporated and the crude product thus obtained was purified by chromatography on silica gel using hexane-EtOAc mixtures of increasing polarity as eluent, to afford 112 mg of the title compound (yield: 48%).

LC-MS (method 1): t_{R}=7.20 min; m/z=407.1 [M+H]⁺.

### Example 11A

### N-Cyclopropyl-3-[N-(2,2-dimethyl-1-oxoindan-5-yl)-N-(2-hydroxyethyl)amino]-4-methylbenzamide

Following a similar procedure to that described in example 11, but using 2-bromoethanol tetrahydropyranyl ether instead of 3-bromopropanol tetrahydropyranyl ether, the title compound was obtained.

LC-MS (method 4): t_{R}=6.21 min; m/z=393.5 [M+H]⁺.

### Example 12

### N-Cyclopropyl-3-[N-(2,2-dimethyl-1-oxoindan-5-yl)-N-(3-(morpholin-4-yl)propyl)amino]-4-methylbenzamide

### a) 3-[N-(5-Cyclopropylaminocarbonyl-2-methylphenyl)-N-(2,2-dimethyl-1-oxoindan-5-yl)amino]propyl methanesulfonate

To a solution of N-cyclopropyl-3-[N-(2,2-dimethyl-1-oxoindan-5-yl)-N-(3-hydroxypropyl)amino]-4-methylbenzamide (90 mg, 0.22 mmol, obtained in example 11) in dry CH₂Cl₂ (2.2 mL), TEA (29 mg, 0.29 mmol) was added and the mixture was cooled to 0° C. Methanesulfonyl chloride (26 mg, 0.23 mmol) was added and the mixture was stirred at room temperature overnight. After dilution with water, the phases were separated. The aqueous phase was reextracted with CHCl₃, the combined organic phases were dried over Na₂SO₄ and the solvent was evaporated to afford 110 mg of the title compound (yield: 97%).

LC-MS (method 1): t_{R}=8.25 min; m/z=485.2 [M+H]⁺.

### b) Title Compound

A mixture of 3-[N-(5-cyclopropylaminocarbonyl-2-methylphenyl)-N-(2,2-dimethyl-1-oxoindan-5-yl)amino]propyl methanesulfonate (110 mg, 0.21 mmol, obtained in section a) and morpholine (37 mg, 0.43 mmol) in acetonitrile (2 mL) was stirred at 70° C. overnight. The solvent was evaporated and the residue was diluted with CHCl₃ and saturated NaHCO₃. The phases were separated, the organic phase was dried over Na₂SO₄ and the solvent was evaporated. The crude product thus obtained was purified by chromatography on silica gel using hexane-EtOAc mixtures of increasing polarity as eluent, to afford 67 mg of the title compound (yield: 62%).

LC-MS (method 1): t_{R}=5.49 min; m/z=476.3 [M+H]⁺.

### Examples 12A-12G

Following a similar procedure to that described in example 12, but using the appropriate amine in step b) instead of morpholine, the compounds in the following table were obtained:

| | | | LC-MS | | |
|---|---|---|---|---|---|
| | | | | t_{R} | m/z |
| Example | Compound name | Amine | Method | (min) | [M + H]⁺ |
| 12A | N-Cyclopropyl-3-[N-(2,2- | Dimethylamine | 4 | 6.69 | 434.6 |
| | dimethyl-1-oxoindan-5-yl)-N- | (The reaction was | | | |
| | (3-dimethylaminopropyl)amino]- | carried out in a sealed | | | |
| | 4-methylbenzamide | tube using THF as the | | | |
| | | solvent) | | | |
| 12B | N-Cyclopropyl-3-[N-(2,2- | 4-(2-hydroxyethyl)- | 4 | 6.43 | 518.4 |
| | dimethyl-1-oxoindan-5-yl)-N- | piperidine | | | |
| | (3-(4-(2-hydroxyethyl)- | | | | |
| | piperidin-1-yl)propyl)amino]-4- | | | | |
| | methylbenzamide | | | | |
| 12C* | 3-[N-(3-(4-Aminopiperidin-1- | 4-(tert- | 4 | 5.85 | 489.5 |
| | yl)propyl)-N-(2,2-dimethyl-1- | butoxycarbonylamino)piperidine | | | |
| | oxoindan-5-yl)amino]-N- | | | | |
| | cyclopropyl-4- | | | | |
| | methylbenzamide | | | | |
| 12D | (R)-N-Cyclopropyl-3-[N-(2,2- | (R)-(+)-Pyrrolidin-3-ol | 4 | 6.03 | 476.5 |
| | dimethyl-1-oxoindan-5-yl)-N- | | | | |
| | (3-(3-hydroxypyrrolidin-1- | | | | |
| | yl)propyl)amino]-4- | | | | |
| | methylbenzamide | | | | |
| 12E | N-Cyclopropyl-3-[N-(2,2- | Piperidin-4-ol | 4 | 6.12 | 490.6 |
| | dimethyl-1-oxoindan-5-yl)-N- | | | | |
| | (3-(4-hydroxypiperidin-1- | | | | |
| | yl)propyl)amino]-4- | | | | |
| | methylbenzamide | | | | |
| 12F | N-Cyclopropyl-3-[N-(2,2- | 2-methoxyethylamine | 4 | 6.31 | 464.5 |
| | dimethyl-1-oxoindan-5-yl)-N- | | | | |
| | (3-(2-methoxyethylamino)propyl)amino]- | | | | |
| | 4-methylbenzamide | | | | |
| 12G | N-Cyclopropyl-3-[N-(2,2- | bis(2- | 4 | 6.11 | 494.6 |
| | dimethyl-1-oxoindan-5-yl)-N- | hydroxyethyl)amine | | | |
| | (3-(bis(2-hydroxyethyl)amino)propyl)amino]- | | | | |
| | 4-methylbenzamide | | | | |

*Compound is obtained as the Boc-protected amine, which is deprotected by stirring with trifluoroacetic acid in CH₂Cl₂ at room temperature overnight.

### Examples 12H-12I

Following a similar procedure to that described in example 12, but starting from example 11A instead of example 11 and using the appropriate amine in step b) instead of morpholine, the compounds in the following table were obtained:

| | | | LC-MS | | |
|---|---|---|---|---|---|
| | | | | t_{R} | m/z |
| Example | Compound name | Amine | Method | (min) | [M + H]⁺ |
| 12H | N-Cyclopropyl-3-[N-(2,2- | 2- | 4 | 6.29 | 450.5 |
| | dimethyl-1-oxoindan-5-yl)-N- | (methylamino)ethanol | | | |
| | [2-[(2- | | | | |
| | hydroxyethyl)methylamino]ethyl]amino]- | | | | |
| | 4-methylbenzamide | | | | |
| 12I* | N-Cyclopropyl-3-[N-(2,2-dimethyl- | 4-(tert- | 4 | 5.76 | 461.5 |
| | 1-oxoindan-5-yl)-N-(2-(piperazin-1- | butoxycarbonyl)piperazine | | | |
| | yl)ethyl)amino]-4-methylbenzamide | | | | |

*Compound is obtained as the Boc-protected piperazine, which is deprotected by stirring with trifluoroacetic acid in CH₂Cl₂ at room temperature overnight

### Example 13

### N-Cyclopropyl-3-(2,2-dimethyl-1-oxoindan-5-ylamino)-4-fluorobenzamide

A solution of 5-bromo-2,2-dimethylindan-1-one (215 mg, 0.9 mmol, obtained in reference example 4) in toluene (8 mL) was refluxed for 30 min under argon and then allowed to cool to room temperature. Palladium acetate (II) (11 mg, 0.05 mmol), (±) 2,2′-bis(diphenylphosphino)-1,1′-binaphthyl (33 mg, 0.05 mmol), cesium carbonate (0.88 g, 2.7 mmol) and 3-amino-N-cyclopropyl-4-fluorobenzamide (0.35 g, 1.80 mmol, obtained in reference example 31) were added. The mixture was inertized with argon and it was heated at 90° C. overnight. The reaction mixture was allowed to cool to room temperature and filtered over a pad of celite. CHCl₃ and water were added, the phases were separated and the organic phase was washed with 3N HCl and dried over Na₂SO₄. The solvent was evaporated and the crude product thus obtained was purified by chromatography on silica gel using hexane-EtOAc mixtures of increasing polarity as eluent, to afford 196 mg of the title compound (yield: 62%).

LC-MS (method 1): t_{R}=7.61 min; m/z=353.1 [M+H]⁺.

### Examples 14-15

Following a similar procedure to that described in example 13, but starting from the appropriate amine in each case, the compounds in the following table were obtained:

| | | | LC-MS | | |
|---|---|---|---|---|---|
| | | | | t_{R} | m/z |
| Example | Compound name | Starting amine | Method | (min) | [M + H]⁺ |
| 14 | N-Cyclopropyl-3-(2,2- | Reference example | 1 | 7.48 | 363.1 [M − H]⁻ |
| | dimethyl-1-oxoindan-5- | 32 | | | |
| | ylamino)-4- | | | | |
| | methoxybenzamide | | | | |
| 15 | 4-Chloro-N-cyclopropyl-3- | Reference example | 1 | 8.16 | 369.3/371.3 |
| | (2,2-dimethyl-1-oxoindan-5- | 33 | | | |
| | ylamino)benzamide | | | | |

### Example 16

### N-Cyclopropyl-3-(2,2-dimethyl-1-oxoindan-5-ylamino)benzamide

Following a similar procedure to that described in example 1, but starting from 3-(2,2-dimethyl-1-oxoindan-5-ylamino)benzoic acid (obtained in reference example 24A), the desired compound was obtained.

LC-MS (method 1): t_{R}=7.50 min; m/z=335.1 [M+H]⁺.

### Example 17

### 2-Cyclopropyl-N-[5-(2,2-dimethyl-1-oxoindan-5-ylamino)-2-methylphenyl]acetamide

Following a similar procedure to that described in example 4, but starting from 5-(3-amino-4-methylphenylamino)-2,2-dimethylindan-1-one (obtained in reference example 29) and cyclopropylacetic acid, the desired compound was obtained.

LC-MS (method 1): t_{R}=8.19 min; m/z=363.3 [M+H]⁺.

### Example 18

### N-Cyclopropyl-3-[N-(2,2-dimethyl-1-oxoindan-5-yl)-N-(2-methoxyacetyl)amino]-4-methylbenzamide

To a solution of N-cyclopropyl-3-(2,2-dimethyl-1-oxoindan-5-ylamino)-4-methylbenzamide (100 mg, 0.29 mmol, obtained in example 2) in CH₂Cl₂ (2 mL), TEA (0.05 mL, 0.34 mmol) was added and the mixture was cooled to 0° C. Methoxyacetyl chloride (34 mg, 0.31 mmol) was added and the mixture was stirred at room temperature overnight and then it was heated at 40° C. for 2 h. Additional portions of TEA and methoxyacetyl chloride were added and the mixture was stirred at 40° C. for another 48 h. The reaction mixture was allowed to cool to room temperature and it was then diluted with CHCl₃ and water. The phases were separated and the organic phase was washed with 2N NaOH and dried over Na₂SO₄. The solvent was evaporated and the crude product obtained was purified by chromatography on silica gel using hexane-EtOAc mixtures of increasing polarity as eluent, to afford 39 mg of the title compound (yield: 32%).

LC-MS (method 3): t_{R}=7.69 min; m/z=421.4 [M+H]⁺.

### Example 19

### 3-[N-Cyclopropanecarbonyl-N-(2,2-dimethyl-1-oxoindan-5-yl)amino]-N-cyclopropyl-4-methylbenzamide

Following a similar procedure to that described in example 18, but starting from N-cyclopropyl-3-(2,2-dimethyl-1-oxoindan-5-ylamino)-4-methylbenzamide (obtained in example 2) and cyclopropanecarbonyl chloride, the desired compound was obtained.

LC-MS (method 4): t_{R}=8.44 min; m/z=417.4 [M+H]⁺.

### Example 20

### 3-(2-Cyclopentyl-1-oxo-2,3-dihydroisoindol-5-ylamino)-N-cyclopropyl-4-methylbenzamide

Following a similar procedure to that described in example 13, but starting from 5-bromo-2-cyclopentyl-2,3-dihydroisoindol-1-one (obtained in reference example 3C) and 3-amino-N-cyclopropyl-4-methylbenzamide (obtained in reference example 35), the desired compound was obtained.

LC-MS (method 4): t_{R}=6.72 min; m/z=390.5 [M+H]⁺.

### Example 21

### N-Cyclopropyl-3-[N-(2,2-dimethyl-1-oxoindan-5-yl)-N-(methanesulfonyl)amino]-4-methylbenzamide

To a suspension of sodium hydride (17 mg 60% in mineral oil, 0.42 mmol) in dry DMF (3 mL), N-cyclopropyl-3-(2,2-dimethyl-1-oxoindan-5-ylamino)-4-methylbenzamide (100 mg, 0.29 mmol, obtained in example 2) and methanesulfonyl chloride (32 μL, 0.42 mmol) were sequentially added and the mixture was heated at 60° C. for 18 h. The reaction mixture was allowed to cool to room temperature and it was then diluted with EtOAc and water. The phases were separated and the organic phase was dried over Na₂SO₄. The solvent was evaporated and the crude product obtained was purified by chromatography on silica gel using hexane-EtOAc mixtures of increasing polarity as eluent, to afford 20 mg of the title compound (yield: 16%).

LC-MS (method 4): t_{R}=7.13 min; m/z=427.2 [M+H]⁺.

### Example 22

### Biological Assay

Inhibition of p38α Enzyme Activity:

Compound stocks in 100% DMSO are first diluted in DMSO to a concentration of 1×10⁻³ up to 3.2×10⁻⁸ M and then further diluted in kinase assay buffer (10 mM Tris-HCl, pH 7.2, 10 mM MgCl₂, 0.01% tween 20, 0.05% NaN₃, 1 mM DTT) to a concentration range of 4×10⁻⁵ up to 1.3×10⁻⁹ M. Of each compound solution 5 μL is transferred into a 384-wells black Optiplate (Packard, 6007279), followed by the addition of 5 μL. of ATP (Boehringer, 519987), 5 μl of Fluorescein-labeled EGFR peptide substrate and 5 μL of active p38α kinase (GST-tagged fusion protein corresponding to full-length human p38α; expressed in E. coli by Upstate, 14-251), all diluted in kinase assay buffer (see final concentrations in Table 1). The mixture is incubated for 2 hours at room temperature (RT). The reaction is stopped by the addition of 60 μL of IMAP binding reagent, which has been diluted 400-fold in IMAP binding buffer (stock concentration 5 times diluted in Milli Q). After incubation for 30 min at RT, FP is measured on an Analyst™ multimode fluorescence plate reader (Molecular Devices) at excitation wavelength of 485 nm and emission wavelength of 530 nm (1 sec/well).

TABLE 1
assay conditions

| Kinase | Final | | Final | ATP final |
|---|---|---|---|---|
| (from Upstate) | concentration | Substrate | concentration | concentration |
| p38α/SAPK2a, | 0.30 U/mL | LVEPLTPSGEAPNQK-(FI) | 240 nM | 20 μM |
| active | | | | |

Data handling is performed as follows: percentage effects are calculated based on no-p38-enzyme-addition as the maximum inhibitory effect and with p38 enzyme addition as the minimum inhibitory effect. In each experiment, individual compound concentrations are tested in duplicate and percentage effect is calculated for each concentration.

Compounds of all examples exhibited more than 50% inhibition at 10 μM in the above assay.

1. A compound of general formula I 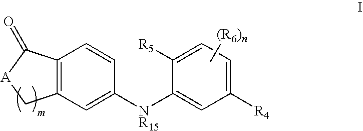
wherein:
A represents CR₁R₂ or NR₃;
R₁ and R₂ independently represent C₁₋₄ alkyl;
R₃ represents —(CH₂)ₚ—Cy¹, or C₁₋₆ alkyl optionally substituted with one or more R₇;
m represents 1 or 2;
R₄ represents —B—R₃;
R₅ represents hydrogen, C₁₋₄ alkyl, halogen or C₁₋₄ alkoxy;
R₆ can be attached to any available carbon atom of the phenyl ring and represents halogen or methyl;
n represents 0 or 1;
B represents —CONR₉—, —NR₉CO— or —NR₉CONR₉—;
R₇ represents hydroxy, C₁₋₄ alkoxy, halogen, —NR₁₀R₁₀ or phenyl optionally substituted with one or more groups selected from C₁₋₄ alkyl, halogen, C₁₋₄ alkoxy, C₁₋₄ haloalkyl and C₁₋₄ haloalkoxy, and additionally two R₇ groups on the same carbon atom can be bonded together to form a —(CH₂)_{q}— group;
R₈ represents C₁₋₆ alkyl or —(CH₂)ₚ—Cy²;
p represents 0, 1 or 2;
q represents 2, 3, 4, 5 or 6;
Cy¹ represents phenyl, heteroaryl, C₃₋₇ cycloalkyl or heterocyclyl, which can all be optionally substituted with one or more R₁₁;
Cy² represents phenyl, heteroaryl or C₃₋₇ cycloalkyl, which can all be optionally substituted with one or more R₁₂;
R₉ and R₁₀ independently represent hydrogen or C₁₋₄ alkyl;
R₁₁ represents halogen, R₁₃, —OR_{13′}, —NO₂, —CN, —COR_{13′}, —CO₂R_{13′}, —CONR₁₄, R_{14′}, —NR₁₄, R_{14′}, —NR_{14′}COR_{13′}, —NR_{14′}CONR_{14′}R_{14′}, —NR₁₄, CO₂R₁₃, —NR_{14′}SO₂R₁₃, —SR_{13′}, —SOR₁₃, —SO₂R₁₃, —SO₂NR_{14′}R_{14′}, or Cy³;
R₁₂ represents C₁₋₄ alkyl, halogen, C₁₋₄ alkoxy, C₁₋₄ haloalkyl, C₁₋₄ haloalkoxy, or Cy³;
R₁₃ represents C₁₋₄ alkyl, C₁₋₄ haloalkyl or C₁₋₄ hydroxyalkyl;
R_{13′} represents hydrogen or R₁₃;
R₁₄ represents C₁₋₄ alkyl or C₁₋₄ hydroxyalkyl;
R_{14′} represents hydrogen or R₁₄;
Cy³ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl and C₁₋₄ haloalkoxy;
R₁₅ represents hydrogen, R₁₆, —COR₁₇, —CONHR₁₇, —SO₂R₁₂ or —COOR₁₇;
R₁₆ represents C₁₋₆ alkyl optionally substituted with one or more groups selected from halogen, —OR_{13′}, —NO₂, —CN, —COR_{13′}, —CO₂R_{13′}, —CONR_{14′}R_{14′}, —NR₁₈R₁₈, —NR_{14′}COR_{13′}, —NR_{14′}CONR_{14′}R_{14′}, —NR_{14′}CO₂R₁₃, —NR_{14′}SO₂R₁₃, —SR_{13′}, —SOR₁₃, —SO₂R₁₃, —SO₂NR_{14′}R_{14′} and Cy⁴;
R₁₇ represents R₁₆ or Cy⁴;
R₁₈ represents hydrogen, C₁₋₄ alkyl, C₁₋₄ hydroxyalkyl or C₁₋₄ alkoxyC₁₋₄alkyl;
Cy⁴ represents phenyl, heteroaryl, C₃₋₇ cycloalkyl or heterocyclyl, which can all be optionally substituted with one or more groups selected from C₁₋₄ alkyl, halogen, C₁₋₄ alkoxy, C₁₋₄ haloalkyl, C₁₋₄ haloalkoxy, hydroxy, C₁₋₄ hydroxyalkyl and —NR₁₉R₁₉; and
R₁₉ represents hydrogen or C₁₋₄ alkyl;
or a salt thereof.

2. A compound according to claim 1 wherein Cy⁴ represents Cy³ and —NR₁₈R₁₈ represents —NR_{14′}R_{14′}.

3. A compound according to claim 1 or 2 wherein A represents CR₁R₂.

4. A compound according to claim 1 or 2 wherein A represents NR₃.

5. A compound according to claim 1 or 2 wherein m is 1.

6. A compound according to claim 1 or 2 wherein R₃ represents —(CH₂)ₚ—Cy¹, C₁₋₆ alkyl or C₁₋₆ hydroxyalkyl.

7. A compound according to claim 6 wherein R₃ represents Cy¹, C₁₋₆ alkyl or C₁₋₆ hydroxyalkyl.

8. A compound according to claim 1 or 2 wherein R₁ is identical to R₂ and both represent methyl.

9. A compound according to claim 1 or 2 wherein R₅ represents hydrogen, methyl, halogen or methoxy.

10. A compound according to claim 1 or 2 wherein B represents —CONR₉— or —NR₉CO—.

11. A compound according to claim 1 or 2 wherein R₁₅ represents hydrogen, R₁₆, —COR₁₇— or —SO₂R₁₇.

12. A compound according to claim 11 wherein R₁₅ represents hydrogen or C₁₋₆ alkyl optionally substituted with one or more groups selected from —OR_{13′}, —NR₁₈R₁₈ and Cy⁴.

13. A compound according to claim 1 selected from:
N-Cyclopropyl-4-methyl-3-(1-oxo-2-phenyl-2,3-dihydroisoindol-5-ylamino)benzamide;
4,N-Dimethyl-3-(1-oxo-2-phenyl-2,3-dihydroisoindol-5-ylamino)benzamide;
N-Cyclopropyl-3-(2-ethyl-1-oxo-2,3-dihydroisoindol-5-ylamino)-4-methylbenzamide;
N-Cyclopropyl-3-[2-(3-hydroxypropyl)-1-oxo-2,3-dihydroisoindol-5-ylamino]-4-methylbenzamide;
N-Cyclopropyl-3-[2-(2-hydroxyethyl)-1-oxo-2,3-dihydroisoindol-5-ylamino]-4-methylbenzamide;
N-Cyclopropyl-3-(2,2-dimethyl-1-oxoindan-5-ylamino)-4-methylbenzamide;
N-Cyclopropylmethyl-3-(2,2-dimethyl-1-oxoindan-5-ylamino)-4-methylbenzamide;
4,N-Dimethyl-3-(2,2-dimethyl-1-oxoindan-5-ylamino)benzamide;
3-(2,2-Dimethyl-1-oxoindan-5-ylamino)-4-methyl-N-phenylbenzamide;
3-(2,2-Dimethyl-1-oxoindan-5-ylamino)-4-methyl-N-(3-pyridyl)benzamide;
N-Benzyl-3-(2,2-dimethyl-1-oxoindan-5-ylamino)-4-methylbenzamide;
3-(2,2-Dimethyl-1-oxoindan-5-ylamino)-4-methyl-N-(2-thiazolyl)benzamide;
3-(2,2-Dimethyl-1-oxoindan-5-ylamino)-4,N,N-trimethyl benzamide;
N-Cyclopropyl-3-(2,2-dimethyl-1-oxo-1,2,3,4-tetrahydronaphthalen-6-ylamino)-4-methylbenzamide;
N-[4-methyl-3-(1-oxo-2-phenyl-2,3-dihydroisoindol-5-ylamino)phenyl]furan-3-carboxamide;
2-Cyclopropyl-N-[4-methyl-3-(1-oxo-2-phenyl-2,3-dihydroisoindol-5-ylamino)phenyl]acetamide;
2-Cyclopropyl-N-[3-(2-(3-hydroxypropyl)-1-oxo-2,3-dihydroisoindol-5-ylamino)-4-methylphenyl]acetamide;
N-[3-(2,2-Dimethyl-1-oxo-1,2,3,4-tetrahydronaphthalen-6-ylamino)-4-methylphenyl]furan-3-carboxamide;
N-[3-(2,2-Dimethyl-1-oxoindan-5-ylamino)-4-methylphenyl]cyclopropylcarboxamide;
2-Cyclopropyl-N-[3-(2,2-dimethyl-1-oxoindan-5-ylamino)-4-methylphenyl]acetamide;
N-[3-(2,2-Dimethyl-1-oxoindan-5-ylamino)-4-methylphenyl]furan-3-carboxamide;
N-[3-(2,2-Dimethyl-1-oxoindan-5-ylamino)-4-methylphenyl]thiophene-2-carboxamide;
2-Chloro-N-[3-(2,2-dimethyl-1-oxoindan-5-ylamino)-4-methylphenyl]isonicotinamide;
N-[3-(2,2-Dimethyl-1-oxoindan-5-ylamino)-4-methylphenyl]-2-(pyrrolidin-1-yl)isonicotinamide;
2-Cyclopropyl-N-[3-(2,2-dimethyl-1-oxo-indan-5-ylamino)phenyl]acetamide;
N-[3-(2,2-Dimethyl-1-oxoindan-5-ylamino)-4-methylphenyl]acetamide;
1-[3-(2,2-Dimethyl-1-oxoindan-5-ylamino)-4-methylphenyl]-3-isopropylurea;
N-Cyclopropyl-3-[N-(2,2-dimethyl-1-oxoindan-5-yl)-N-methylamino]-4-methylbenzamide;
N-Cyclopropyl-3-[N-(2,2-dimethyl-1-oxoindan-5-yl)-N-(3-hydroxypropyl)amino]-4-methylbenzamide;
N-Cyclopropyl-3-[N-(2,2-dimethyl-1-oxoindan-5-yl)-N-(2-hydroxyethyl)amino]-4-methylbenzamide;
N-Cyclopropyl-3-[N-(2,2-dimethyl-1-oxoindan-5-yl)-N-(3-(morpholin-4-yl)propyl)amino]-4-methylbenzamide;
N-Cyclopropyl-3-[N-(2,2-dimethyl-1-oxoindan-5-yl)-N-(3-dimethylaminopropyl)amino]-4-methylbenzamide;
N-Cyclopropyl-3-[N-(2,2-dimethyl-1-oxoindan-5-yl)-N-(3-(4-(2-hydroxyethyl)-piperidin-1-yl)propyl)amino]-4-methylbenzamide;
3-[N-(3-(4-Aminopiperidin-1-yl)propyl)-N-(2,2-dimethyl-1-oxoindan-5-yl)amino]-N-cyclopropyl-4-methylbenzamide;
(R)—N-Cyclopropyl-3-[N-(2,2-dimethyl-1-oxoindan-5-yl)-N-(3-(3-hydroxypyrrolidin-1-yl)propyl)amino]-4-methylbenzamide;
N-Cyclopropyl-3-[N-(2,2-dimethyl-1-oxoindan-5-yl)-N-(3-(4-hydroxypiperidin-1-yl)propyl)amino]-4-methylbenzamide;
N-Cyclopropyl-3-[N-(2,2-dimethyl-1-oxoindan-5-yl)-N-(3-(2-methoxyethylamino) propyl)amino]-4-methylbenzamide;
N-Cyclopropyl-3-[N-(2,2-dimethyl-1-oxoindan-5-yl)-N-(3-(bis(2-hydroxyethyl)amino)propyl)amino]-4-methylbenzamide;
N-Cyclopropyl-3-[N-(2,2-dimethyl-1-oxoindan-5-yl)-N-[2-[(2-hydroxyethyl)methylamino]ethyl]amino]-4-methylbenzamide;
N-Cyclopropyl-3-[N-(2,2-dimethyl-1-oxoindan-5-yl)-N-(2-(piperazin-1-yl)ethyl)amino]-4-methylbenzamide;
N-Cyclopropyl-3-(2,2-dimethyl-1-oxoindan-5-ylamino)-4-fluorobenzamide;
N-Cyclopropyl-3-(2,2-dimethyl-1-oxoindan-5-ylamino)-4-methoxybenzamide;
4-Chloro-N-cyclopropyl-3-(2,2-dimethyl-1-oxoindan-5-ylamino)benzamide;
N-Cyclopropyl-3-(2,2-dimethyl-1-oxoindan-5-ylamino)benzamide;
2-Cyclopropyl-N-[5-(2,2-dimethyl-1-oxoindan-5-ylamino)-2-methylphenyl]acetamide;
N-Cyclopropyl-3-[N-(2,2-dimethyl-1-oxoindan-5-yl)-N-(2-methoxyacetyl)amino]-4-methylbenzamide;
3-[N-Cyclopropanecarbonyl-N-(2,2-dimethyl-1-oxoindan-5-yl)amino]-N-cyclopropyl-4-methylbenzamide;
3-(2-Cyclopentyl-1-oxo-2,3-dihydroisoindol-5-ylamino)-N-cyclopropyl-4-methylbenzamide; and
N-Cyclopropyl-3-[N-(2,2-dimethyl-1-oxoindan-5-yl)-N-(methanesulfonyl)amino]-4-methylbenzamide.

14. A pharmaceutical composition which comprises a compound of formula I according to claim 1 or 2 or a pharmaceutically acceptable salt thereof and one or more pharmaceutically acceptable excipients.

15. A method of treating or preventing a disease mediated by p38 in a subject in need thereof which comprises administering to said subject an effective amount of a compound of formula I according to claim 1 or 2 or a pharmaceutically acceptable salt thereof.

16. A method according to claim 15, wherein the disease mediated by p38 is selected from immune, autoimmune and inflammatory diseases, cardiovascular diseases, infectious diseases, bone resorption diseases, neurodegenerative diseases, proliferative diseases and processes associated with the induction of cyclooxygenase-2.
